(19) United States
(12) Patent Application Publication
(10) Pub. No.: US 2011/0166038 A1
(43) Pub. Date: Jul. 7, 2011

(54) SCREENING METHODS FOR HEAT-SHOCK RESPONSE MODULATORS

(75) Inventors: Bin Zhang, San Diego, CA (US); Shi Chung Ng, San Diego, CA (US); Qingyan Au, Anaheim, CA (US)

(73) Assignee: SCREENING METHODS FOR HEAT-SHOCK RESPONSE MODULATIORS

(21) Appl. No.: 12/994,703

(22) PCT Filed: Jun. 3, 2009
(86) PCT No.: PCT/US09/03401

§ 371 Date: Jan. 21, 2011

(30) Foreign Application Priority Data
Jun. 3, 2008 (US) 61130945
Oct. 10, 2008 (US) 61194984

(51) Int. Cl. C40B 30/06; C40B 60/00; C07C 61/29

(52) U.S. Cl. 506/10; 506/33; 562/403

(57) ABSTRACT

High-throughput methods are provided for quantitatively measuring the modulation of heat shock protein (HSP) expression in a cell by exposing the cell to at least one stress and measuring cellular stress responses. High-throughput methods for identifying modulators (activators or inhibitors) of HSP or HSF expression in a cell by treating the cell with an agent, such as a compound or composition, exposing the cell to a stress, and measuring responses of the cell to the stress in the presence or absence of the agent are also provided. Devices useful in performing high-throughput methods of the invention, and modulators identified using such methods, are also provided.

### RELATED APPLICATIONS

This application claims the benefit of U.S. Provisional Patent Applications 61/130,945, filed Jun. 3, 2008, and 61/194,984, filed Oct. 1, 2008, the specifications of which are incorporated by reference herein in their entirety.

### BACKGROUND OF THE INVENTION

Heat shock proteins (HSPs) are essential cellular proteins for maintenance of homeostasis and survival against various physiological and environment insults. See Voellmy et al., Adv. Exp. Med. Biol., 594:89-99 (2007). Members of this multigene super-family are designated by their molecular size and relevant function, including HSP110, HSP90, HSP70, HSP60, HSP40 and small heat shock proteins. HSPs function as molecular chaperones to either assist misfolded proteins to restore their conformations and cellular function or guide damaged proteins to proteosome directed degradation. See, e.g., Hendrick et al., Annu. Rev. Biochem., 62:349-384 (1993); Riordan et al., Nat. Clin. Pract. Nephrol., 2:149-156 (2006). HSP expression is rapidly induced by heat shock transcription factor (HSF), specifically HSF1, the prototype regulator that activates HSP gene transcription through binding heat shock element (HSE) sequences in the promoter region regulated genes. See Pirkkala et al., FASEB J., 15:1118-1131 (2001). Because activation of pathways involved in heat shock response is a common cellular reaction to cellular stress, such as stress-related protein misfolding, small molecules that can modulate cellular heat shock response are of great interest for use, e.g., as therapeutic tools for treatment and prevention of a broad range of clinical indications that involve in some aspect the activation or repression of cellular heat shock response including, for example, cancer, ischemia, wound healing and neurodegenerative diseases. In recent years, a number of compounds that modulate HSF1 and HSPs have been discovered and some of them are currently in clinical trials. See, e.g., Powers et al., FEBS Letters, 581:3758-3769 (2007).

HSF1 both positively and negatively regulates genes involved in cellular stress response pathways, by direct and indirect mechanisms. The diverse activities of HSF1 correlate in part with its ability to form multimers having different properties, such as different binding affinities for DNA and protein factors, than do HSF1 monomers. Under normal growth conditions, HSF1 has been shown to reside in the cytoplasm and nucleus of the cell in a relatively inactive, monomeric form. In stress-stimulated cells (e.g., cells exposed to heat shock, heavy metals or amino acid analogs), HSF1 undergoes trimerization with enhanced DNA binding capacity and altered protein interaction profiles. The activity is further elevated through certain sites of inducible phosphorylation. HSF1 not only controls up-regulation of HSP70 mRNA transcription, but also facilitates export of stress-induced HSP70 mRNA by interaction with the nuclear pore-associated TPR protein. See Skaggs et al., J. Biol. Chem., 282(47):33902-33907 (2007).

Interestingly, HSF1 redistributes from a widely diffuse pattern to discrete HSF1 containing granules within the nuclei of stressed cells. See Cotto et al., J. Cell Sci., 110(23):2925-2934 (1997). These stress granules are reported to be large and irregularly shaped, and appear to be primarily located through direct DNA-protein interactions with satellite III repeats. See Jolly et al., J. Cell Biol., 156(5); 775-781 (2002); Jolly et al., J. Cell Biol., 164(1):25-33 (2004).

HSP70 protein is one of the most important families of molecular chaperones. This family contains eight highly homologous chaperone proteins with overlapping and distinct functions. See Daugaard et al., FEBS Lett., 581(19):3702-3710 (2007). The major function of HSP70 is to provide cytoprotection against stress induced protein misfolding or denaturation. In addition, constitutively expressed HSP70 protein also plays important house-keeping roles in non-stressed cells. Following heat shock, there is a significant increase in HSP70 expression and a majority of the newly synthesized HSP70 protein rapidly migrates from the cytoplasm into the nucleus of the cell. Using GFP fusions to HSP70, Zeng et al. reported that, upon cellular stress, GFP-HSP70 levels are significantly increased in the nucleus and become highly concentrated in the nucleoli, designated as HSP70 granule. See Zeng et al., J. Cell Sci., 117(21):4991-5000 (2004). There is also a negative feedback loop to balance the expression of HSPs. HSP90 and HSP70 protein in the multichaperone complex interacts with HSF1 to repress its activity. Stress induced misfolded proteins disrupt the interaction and release HSF1 for transcriptional activation.

A great deal of effort has been made to screen for therapeutically active small molecules targeting HSF1/HSPs in the heat shock response. Many compounds have been identified as HSF1 modulators via direct HSF1 activation (celastrol), HSP90 inhibition (radicicol, 17-AAG), inflammatory mediation (arachidonic acid, terracyclic acid A), proteosome inhibition (MG-132), heat shock response inhibition (KNK437, quercetin), and HSF1/HSP70 coinduction (arimoclomol, bimoclomol). See Westerheide et al., J. Biol. Chem., 280(39):33907-33100 (2005). In recent years, two major schemes have emerged for targeted therapeutics. On the one hand, inhibition of HSP90 has provided an avenue for anticancer therapy because HSP90 stabilizes several key kinases involved in malignant transformation. See Whitesell et al., Curr. Cancer Drug Targets, 3:349-358 (2003). Several HSP90 inhibitors are currently in clinical trials and one such inhibitor, 17-AAG, has shown clear antitumor activity with manageable toxicity profiles. On the other hand, up-regulation via small molecules of HSPs, and especially HSP70, has shown great therapeutic value in diseases, conditions and disorders in which accumulation of misfolded proteins is apparent and appears to contribute to unwanted symptoms. Importantly, some compounds in this category, such as arimoclomol, have no inductive effect on HSP70 and other chaperone proteins in normal cells, but do activate the enhanced chaperone induction in stressed cells (so-called “coinduction” or “amplification”). Identification of other co-inductive compounds will likely be valuable therapeutic agents and may have fewer adverse side effects than agents which activate HSP70 in normal and stress induced cells. See Soti et al., Br. J. Pharmacol., 146(6):769-780 (2005).

In view of the above, it would be advantageous to develop an assay to identify agents, e.g., small molecules, that regulate (induce, coinduce, amplify, repress or diminish) HSF1/HSP activities in cellular stress response pathways for diagnostic or therapeutic uses in targeted chaperone therapies.

### SUMMARY OF THE INVENTION

In one aspect, the present invention provides a high-throughput method for quantitatively measuring the modulation of heat shock protein (HSP) expression in a cell by treating the cell with a stress and measuring cellular stress responses by a number of variables, and especially, a combination of one or more variables relating to HSF granule (e.g., HSF1, HSF2, HSF3, or HSF4 granule) formation and the characteristics of the granules that form upon cellular stress. In certain embodiments, the combination is of two or more variables. Certain preferred variables relating to HSF granules, such as HSF1 granules, that correlate with cellular stress response may be selected from one or more, and sometimes two or more, of the following: granule count, the coefficient of variability (CV) of nuclear intensity, granule area, granule intensity; and ratio of granule intensity to background intensity. In certain embodiments, when a first variable is granule count, it is measured in combination with a second variable selected from: the coefficient of variability (CV) of nuclear intensity, granule area, granule intensity; and ratio of granule intensity to background intensity. Additional variables in any combination may optionally be measured.

In another aspect, the present invention provides a high-throughput method for identifying modulators (activators or inhibitors) of heat shock protein (HSP) expression in a cell by treating the cell with a candidate agent, such as a candidate compound or candidate composition, exposing the cell to a stress, and measuring responses of the cell to the stress in the presence or absence of the agent. Cellular stress responses may be measured by measuring one or more variables, and especially a combination of two or more variables, relating to granule formation, such as HSP and/or HSF (e.g., HSF1) granule formation, and/or the characteristics of the granules that form upon cellular stress. Certain preferred variables relating to HSP or HSF granules, such as HSF1 granules, that correlate with cellular stress response may be selected from the following: granule count; the coefficient of variability (CV) of nuclear intensity; granule area, granule intensity, and ratio of granule intensity to background intensity. In certain embodiments, when a first variable is granule count, it is measured in combination with a second variable selected from: the coefficient of variability (CV) of nuclear intensity, granule area, granule intensity; and ratio of granule intensity to background intensity. Additional variables in any combination may optionally be measured.

In yet another aspect, the present invention provides a high-throughput method for quantitatively measuring transcriptional activity of HSF, such as HSF1, in a cell by treating the cell with a stress, and further, for identifying modulators (activators or inhibitors) of HSF activity, such as HSF1 activity, in a cell using assays and methods described above. Methods for modulating levels of cellular stress to optimize selection of activators and inhibitors are provided. Selection of HSP and/or HSF granule variables, types of cells, stresses and other variables associated with fidelity and reproducibility of the high-throughput methods of the invention are discussed in detail below.

### BRIEF DESCRIPTION OF THE DRAWINGS

FIGS. 1A-D show granulate formation of HSF1 and HSP70 (A and C) in the nucleus of cultured HeLa cells after heat shock at 41° C. for 2 hours when untreated or pretreated one hour prior to heat shock with 2 μM celastrol (A and C) in DMSO or 0.33% DMSO (B and D) as described in Example 1.

FIGS. 2A-D show the quantification of nuclear HSF1 granules by granule count and nuclear intensity CV (A and B) and quantification of nuclear HSP70 granules by granule count and granulate total area (C and D) from Example 1. The differences of the mean values were compared by the Student's t test. A p value <0.01 (**) was considered statistically significant.

FIG. 3 shows an evaluation of high content screening (HCS) granule assay performance with samples treated with 2 μM celastrol one hour prior to heat shock as positive controls and samples treated with 0.33% DMSO as negative controls.

FIGS. 4A-B show a dose dependent study with EC₅₀ values of HSF1 nuclear intensity CV (A) and HSP70 granule area (B) for celastrol (positive control) and Compound A (a new modulator identified by the present HCS method) as described in Example 1.

FIG. 5 shows a comparison of HSF1 induction kinetics in cells over a 6 hour recovery period after pretreatment with 10 μM of Compound A or with 1 μM celastrol (positive control) as described in Example 1.

FIG. 6 shows data from experiments in which HeLa cells were treated individually with 480 different test compounds 30 minutes before 41° C. heat shock for 2 hours with no recovery time against a 2 μM celastrol positive control (▪) and a DMSO negative control ().

FIG. 7 shows an MTS cell death assay adopted to evaluate cytoprotective effects of Compound A under non-oxygen glucose deprivation (OGD) stress and OGD stress, as described in Example 2. SH-SY5Y cells were treated with 0.33% DMSO or 2.5 μM Compound A in DMSO for one hour before OGD for 28 hours followed by immediate MTS assay. Data was presented as the average of three independent experiments. A p value <0.01 (**) was considered statistically significant.

FIG. 8 shows an MTS cell death assay adopted to evaluate cytoprotective effects of Compound A under rotenone induced mitochondrial stress, as described in Example 3. SH-SY5Y cells were treated for 1 hour with DMSO and 2.5 μM Compound A before 100 nM rotenone treatment for 24 hours followed by immediate MTS assay. Data was presented as the average of three independent experiments. A p value <0.01 (**) was considered statistically significant.

FIG. 9 shows HSF1 granule data in a comparison of cells treated with stress by elevated temperature at either 39° C. or 41° C. for 2 hours with no recovery time.

FIG. 10 shows that the observed CV values are greater than 25% for HSP70 granule count in cells treated with DMSO and screened at an elevated temperature of 43° C. for 1 hour with no recovery time or with a 2 hour recovery time. These data suggest that values for HSF1 granule count at both conditions are closer than desirable to that of the positive control value, and at 43° C., the HCS assay detection window is significantly smaller than at 41° C.

FIG. 11 shows HSF1 and HSP70 granule count evaluation for 0.33% DMSO-treated cells exposed to a 41° C. elevated temperature stress for 2 hours with no recovery period.

FIG. 12 shows a HSF1 CV nuclear intensity and HSP70 granule area evaluation for 0.33% DMSO-treated cells exposed to a 41° C. elevated temperature stress for 2 hours with no recovery period.

FIG. 13 illustrates the response from compound B (a new modulator identified by the present HCS method) at various time points in a tunicamycin induced ER stress model as described in Example 6.

FIG. 14 shows a HSF1 granule count evaluation as a function of increasing concentrations (μM) of celastrol (positive control) and Compound A (test compound) in cells that have not been exposed to elevated temperature stress (e.g., 37° C. for 3 hours). The data show that at concentrations of 1.25-5.0 μM, celastrol significantly stimulates HSF1 positive granule formation in normal (non-heat shocked) cells, whereas Compound A does not.

FIG. 15 shows percent inhibition of HSP90 ATPase activity in cells treated with 10 μM radicicol (control) or 50 μM of one of nine tested compounds, as described in Example 8. The results illustrate that compounds identified as positive hits in the HCS assay do not significantly inhibit the ATPase activity of HSP90.

FIG. 16 provides a strategy for screening compounds for lead development using a primary HSF1/HSP70 granule assay and secondary MG-132 and MTS assays to identify cytoprotection and cytotoxicity, respectively.

FIG. 17A represents a compilation of data (4,000 compounds) showing HSF1 granule positive cells on the x-axis, viable cells from the MG-132 assay on the y-axis and inhibition of viable cells from the MTS assay on the z-axis while the size and shading of the representative spheres correspond to HSP70 and HSF1 granule positive cells respectively.

FIG. 17B represents data from the HSF1 granule screen (4,000 compounds) with a threshold of 20% for HSF1 granule positive cells and square shading corresponds to HSP70 granule positive cells.

FIG. 17C represents data from the HSP70 granule assay with a threshold of 30% for HSP70 granule positive cells and the square shading corresponds to HSF1 granule positive cells.

FIG. 17D illustrates data from the MG-132 assay with a threshold of 30% for percentage increase in viable cells (compared to DMSO), and the square shading corresponds to HSF1 granule positive cells.

FIG. 17E illustrates a compilation of the MG-132 assay data and the MTS assay data while the sphere size corresponds to HSP70 granule data and the shading corresponds to HSF1 granule data.

FIG. 18 shows a 384-well plate evaluation of HeLa cells pre-treated with 0.33% DMSO (♦) and 2 μM celastrol (▪) and subsequently heat shocked at 41° C. for 2 hours with no recovery time (R0) using HSF1 granule count.

FIG. 19 shows a Western blot and bar chart of HeLa cells transfected with 25 nM of HSF1 siRNA, scramble siRNA (control) or GAPDH siRNA (transfection control) for 48 h followed by 43° C. heat shock for 2 hours or non-heat shock treatment.

FIG. 20 illustrates granule formation in HeLa cells treated with 41° C. heat shock for 2 hours or non-heat shock treatment transfected with 25 nM of HSF1 siRNA and scramble siRNA for 48 hours followed by treatment with 25 μM of Compound B (CYT492) or a DMSO control before the treatment.

FIG. 21 shows cell count of HeLa cells transfected with 25 nM of HSF1 siRNA or scramble (non-target) siRNA, demonstrating minimal cytotoxic effects.

FIGS. 22A-B illustrate an siRNA knockdown of HSF1 in SK-N-SH cells with 10, 25 or 50 nM of HSF1-specific siRNA, GAPDH siRNA (control) or scramble siRNA (control) for 48 hours (A) and 72 hours (B) and the corresponding Western blot looking at corresponding protein expression levels.

FIG. 22C shows a Western blot illustrating the effects on HSP70 protein expression after HSF1 knockdown for 48 hours with 10, 25 or 50 nM of HSF1 specific siRNA compared to GAPDH (transfection control) and scramble (control) siRNAs.

FIGS. 23A-D show HSF1 dependent cytoprotection of SK-N-SH cells in the MG-132 assay (Example 5) when treated with 50 nM HSF1 siRNA or scramble siRNA for 48 hours following pretreatment with CYT 2239 (A), CYT 2244 (B), CYT2282 (C) or CYT 2532 (D).

FIG. 24 illustrates the inhibition of HSF1 granule formation in HeLa cells treated with 10 nM, 100 nM, 1 μM and 10 μM concentrations of triptolide after a 43° C. heat shock for 1, 2, 3, or 4 hours using HSF1 granule count with 5 granules/nucleus as the threshold.

FIGS. 25A-D show the reduction of HSP70 expression of HeLa cells treated with 1 triptolide (♦), 10 μM CYT 975 (▪), 10 μM CYT 1563 (▴), or 10 μM CYT 1590 () at 43° C. heat shock for 1, 2, 3, or 4 hours with 0, 5 and 7 hours recovery time using total HSP70 nuclear and cell intensity as the threshold.

FIGS. 26A-D show an evaluation of HSF1 granule count at 43° C. heat shock for (A) 2 hours and no recovery time (R0); and (B) 4 hours and 4 hours recovery time; and an evaluation of HSP70 cellular intensity CV at 43° C. heat shock for (C) 2 hours and 4 hours recovery time; and (D) 4 hours and 4 hours recovery time; in HeLa cells treated with 0.33% DMSO, triptolide (1 μM) or CYT 1563 (10 μM).

FIG. 27 shows a 384-well plate evaluation of HeLa cells treated with 0.33% DMSO and CYT 1563 (10 μM) and subsequently heat shocked at 43° C. for 2 hours with no recovery time (R0) using HSF1 granule count.

### DETAILED DESCRIPTION OF THE INVENTION

### Definitions

For convenience, certain terms employed in the specification, examples, and claims, are collected and defined herein. Unless defined otherwise, all technical and scientific terms used herein have the same meaning as commonly understood by one of ordinary skill in the art.

The term “coefficient of variability (CV) of nuclear intensity” as used herein refers to the difference in color intensity of granules within the nucleus of a cell as measured by imaging with an instrument capable of detecting color labels, such as fluorescent labels.

The term “granule” as used herein refers to an elevated nuclear concentration of HSP, HSF, or other HSP cofactor in the nucleus, wherein the elevated nuclear concentration is associated with elevated levels of HSP or molecular chaperone expression or HSF transcription. Preferably, these granules are detected by imaging with an instrument capable of detecting color labels, such as fluorescent labels. The term “granule” may also be known to one of ordinary skill in the art as a “spot,” “dot,” or “grain” that is associated with elevated levels of HSP or molecular chaperone expression or HSF transcription.

The term “granule count” as used herein refers to the number of granules detected in a cell sample. In some instances, granule count can be measured by counting the number of granules with the naked eye. In other embodiments, granule count is determined by the sensitivity settings and resolution capability of an imaging instrument and the accompanying software. In some embodiments, granule count of HSP or HSF may average from 2 to 30 granules per cell, for example, from 3 to 10 granules per cell.

The term “granule intensity” as used herein refers to the color intensity of granules within the nucleus of a cell as measured by imaging with an instrument capable of detecting color labels, such as fluorescent labels.

The term “granule size” as used herein refers to the detectable size of a granule, such as “granule area,” “granule diameter,” or “granule volume” as measured by an imaging instrument. Typically, granule size is determined by the sensitivity settings and resolution capability of an imaging instrument and the accompanying software. In some instances, granules may have average diameters from about 0.01 to about 20 μm², for example, from about 0.1 to about 10 μm², such as from about 0.2 to about 5 μm². In some instances, granules may have average volumes from about 0.01 to about 100 μm³, for example, from about 0.1 to about 50 μm³, such as from about 1 to about 20 μm³.

The term “maximum stress response” as used herein refers to the response of a cell to a maximum level of stress. The maximum level of stress is the level of applied stress above which the cellular response to the stress does not change. For example, when the stress is elevated temperature (heat shock), the corresponding maximum stress response is the “maximum heat shock response,” which refers to the response of the cell to a “maximum level of heat shock,” i.e., a temperature above which the cellular response to heat shock is unchanged. Other examples of maximum levels of stress include maximum amounts of concentrations of metals, chemical toxicants, oxygen, etc. that induce stress responses in cells.

The term “mild stress” as used herein refers to conditions that provide a sub-maximal stress response in relation to a preconditioning stress. For example, an elevated temperature that is below the maximum heat shock response inducing temperature but which still induces a stress response in a cell is a “mild heat shock” condition. Other examples of mild stress conditions include sub-maximal amounts of concentrations of metals, chemical toxicants, oxygen, etc. that induce stress responses in cells.

The term “modulating” refers to the action of a compound or composition described herein to produce a change in a biological pathway or in the activity or function of a given biological macromolecule, such as a protein, such as HSP, HSF, or a nucleic acid element, such as HSE. In some embodiments, modulating includes inhibiting or antagonizing a biological pathway or inhibiting, antagonizing, or decreasing the activity of a biological macromolecule. In other embodiments, modulating includes promoting or agonizing a biological pathway or promoting, agonizing, or increasing the activity of a biological macromolecule. For example, in certain embodiments, modulating includes producing a change in the transcriptional activity of a biological pathway or transcription factor, such as HSF.

The term “small molecule” as used herein refers to an organic compound having a molecular weight less than about 2500 amu (atomic mass unit), preferably less than about 2000 amu, even more preferably less than about 1500 amu, still more preferably less than about 1000 amu, or most preferably less than about 750 amu. Such molecules typically are composed primarily of two or more of carbon and hydrogen atoms and may include one or more occurrences of oxygen and nitrogen. Such molecules may also include one or more occurrences of sulfur, phosphorus, and halogen (such as fluorine, chlorine and bromine), although other known atoms may also be employed. Suitable small molecules used in the present methods may be synthetic or naturally occurring and may be commercially available in diverse chemical libraries. In some instances, small molecules used in the present methods include hydroxylamine compounds.

The term “stress” as used herein refers to a physiological stress which would be understood by one of ordinary skill in the art to be a condition or factor affecting the cell that would induce the “stress response” of the cell. Examples of inducers of stress include elevated temperatures, metals, chemical toxicants, oxygen provision and deprivation, etc.

The term “stress response” as used herein refers to an increase in HSP expression and/or HSF transcription in response to exposure to a cellular stress. For example, a response to an elevated temperature stress is a heat shock response.

The term “sub-lethal stress” as used herein refers to a stress that induces a stress response of the cell without causing the death of the cell.

The term “submaximal stress response” as used herein refers to a stress response below that produced by the maximum stress level but above that produced by the precondition stress, for example, a heat shock response below the heat shock response produced by the maximum heat shock inducing temperature but above that of the precondition stress inducing temperature.

The term “treatment” as used herein refers to an amelioration in the clinical condition of the subject and does not indicate that a cure is achieved.

Each patent and non-patent publication referred to herein is incorporated herein by reference in its entirety.

### Embodiments

### Methods for High-Throughput Quantification of HSP or HSF Expression

Heat shock induced stress granule formation has been reported and noted to correlate with heat shock response. See Cotto et al., supra; Zeng et al., supra. See also Zaarur et al., Cancer Res., 66(3):1783-1791 (2006). To date, however, there has been no assay reported that can directly measure activation of HSF1/HSP in a high throughput format. The fact that both HSF1 and HSP70 form stress granules after heat shock could provide useful cellular markers for quantifying cellular stress activation if methods for accurately and reproducibly quantifying such markers could be developed. To address this need, the present invention provides image-based high content screening (HCS) that is capable of accurately quantifying stress granule formation in a cell which correlates with HSF1/HSP activity. The multi-parametric nature of HCS is particularly useful for analyzing complex cellular networks and biological mechanisms with a reasonable throughput. See Johnston, P. A., High Content Screening, 25-42, (2008); Zhang et al., J. Biomol. Screen, 13(10):953-9 (2008). Several parameters of granule formation have been selected herein, including granule count, total granule area, granule intensity, ratio of granule intensity to background intensity, and the CV of nuclear intensity for quantification of stress activated HSF, such as HSF1, and/or HSP, such as HSP70.

Accordingly, in some embodiments, the present invention provides a high-throughput method for quantitatively measuring the modulation of HSP expression in a cell by exposing the cell to a stress and measuring cellular stress responses by one or more variables, or a combination of two or more variables, relating to HSP and/or HSF granule formation (e.g., HSF1 granule formation) and the characteristics of the granules that form upon cellular stress. Certain preferred variables relating to HSP and/or HSF granules (e.g., HSF1 granules) that correlate with cellular stress response may be selected from one or more, and preferably two or more, of the following: granule count, the CV of nuclear intensity, granule area, granule intensity; and ratio of granule intensity to background intensity. In certain embodiments, when a first variable is granule count, it is measured in combination with a second variable selected from: the CV of nuclear intensity, granule area, granule intensity; and ratio of granule intensity to background intensity. Additional variables in any combination may optionally be measured.

In yet another aspect, the present invention provides a high-throughput method for quantitatively measuring transcriptional activity of HSF, such as HSF1, comprising exposing a cell to a stress and measuring cellular stress responses by one or more variables, or a combination of two or more variables, relating to HSP and/or HSF granule formation (e.g., HSF1 granule formation) and the characteristics of the granules that form upon cellular stress. Certain preferred variables relating to HSP and/or HSF granules that correlate with cellular stress response may be selected from one or more, and preferably two or more, of the following: granule count, the CV of nuclear intensity, granule area, granule intensity; and ratio of granule intensity to background intensity. In certain embodiments, when a first variable is granule count, it is measured in combination with a second variable selected from: the CV of nuclear intensity, granule area, granule intensity, and ratio of granule intensity to background intensity. Additional variables in any combination may optionally be measured.

### Methods for Identifying Modulators of HSP Expression

In another aspect, the present invention provides a high-throughput method for identifying a modulator (an activator or inhibitor) of HSP expression in a cell by treating the cell with an agent that is a putative modulator (i.e., a candidate agent), such as a candidate compound or candidate composition comprising an active compound; exposing treated and untreated control cells to a stress; and measuring responses of the cells to the stress in the continued presence of or upon a timed withdrawal of the putative modulator. Additionally, in another embodiment, the present invention provides a high-throughput method for identifying a modulator (an activator or inhibitor) of HSF, such as HSF1, expression in a cell by treating the cell with an agent that is a putative modulator (i.e., a candidate agent), such as a candidate compound or candidate composition comprising an active compound; exposing treated and untreated control cells to a stress; and measuring responses of the cells to the stress in the continued presence of or upon a timed withdrawal of the putative modulator. Cellular stress responses may be measured by measuring one or more variables, and especially a combination of two or more variables, relating to HSP and/or HSF granule (e.g., HSF1 granule) formation and the characteristics of the granules that form upon cellular stress. Certain preferred variables relating to HSP and/or HSF granules that correlate with cellular stress response may be selected from the following: granule count, the CV of nuclear intensity, granule area, granule intensity, and ratio of granule intensity to background intensity. In certain embodiments, when a first variable is granule count, it is measured in combination with a second variable selected from: the CV of nuclear intensity, granule area, granule intensity, and ratio of granule intensity to background intensity. Additional variables in any combination may optionally be measured.

In some embodiments, the putative modulator of HSP expression is an agent that consists of or comprises a polypeptide sequence. In other embodiments, the agent consists of or comprises a small molecule. In still other embodiments, the agent consists of or comprises a nucleic acid moiety, e.g., DNA, RNA, or a combination thereof. In certain embodiments, the nucleic acid moiety is or produces an inhibitory RNA, such as an siRNA, shRNA, miRNA, or other small nucleic acid molecule that mediates RNA interference or that otherwise regulates transcription, processing, or translation of RNA, including mRNA.

In certain embodiments, the putative modulator is administered to the cell for a period of time before the cell is exposed to the stress. Suitable periods of time may be selected by the skilled practitioner depending on the types of agents being tested (and taking into consideration how quickly they may act on the cellular response); the mitotic or other cellular growth states of the tested cells, and the like. Modulators of stress response, such as heat shock response, that work or work better with a pre-treatment stage may be useful in preventative therapeutic methods of the invention. Cells may be pretreated with putative modulators for, e.g., days, hours, minutes or seconds (and fractions thereof) before the cells are exposed to the selected stress. The putative modulators may optionally be removed from the cells at various times after the stress and induction of stress response.

The High Content Screening (HCS) granule assay of the present invention enables the rapid quantification of variables used in the high-throughput method, preferably in an automated fashion. Accordingly, in certain embodiments, the HCS is automated and is capable of screening a large number of compounds with a reasonably fast throughput. In certain embodiments, the HCS is capable of screening about 2,000 to 10,000 compounds per day. In some embodiments, the HCS granule assay makes use of advanced imaging software to significantly improve complicated image segmentation and high speed data processing. One such embodiment exemplified herein is referred to as the “Master Chaperone Regulator Assay” or “MaCRA” (see also Zhang et al., J. Biomol. Screen, 13(10):953-9 (2008). MaCRA is a cell image-based screening tool that enables rapid, quantifiable screening of large numbers of small molecule compounds to identify potential drug candidates that modify the activity of HSF1. Modulators of HSF1 are expected to control entire groups of molecular chaperone proteins that repair or degrade toxic misfolded proteins present in diseased cells. Certain other types of HSF1 modulators are expected to affect apoptosis, cytotoxicity and growth regulation of cancer or tumor cells. Evaluation of certain of the compounds identified thus far in MaCRA screens, as exemplified herein, shows that they exhibit cytoprotective properties in cell culture models of disease.

One example of imaging software for the methods of the present application includes Multi Target Analysis (MTA) module from Workstation software (GE Healthcare), which provides a high-speed measurement of intracellular granules with a comprehensive report of granule count, granule area, granule intensity and CV of nuclear intensity. Other imaging systems and software may be adopted for use in the assays and methods of the invention. In certain embodiments, the imaging system and software stores information on the assay conditions and results for each individual compound tested in any given assay, the information stored in digital formal and entered into a database for compiling larges data sets that can be used as comparators to other test compounds. Compounds may thus be classified into types and subtypes according to their performance in one or a combination of assays, such classifications later being used for understanding structure-function relationships and for predictive chemistry and biology.

In certain embodiments, the present methods, such as the HCS granule assay, may be used to screen the response of different cells to one or more different stress conditions. The stress that is used in any of the methods of the present invention may be selected from, but is not limited to, elevated temperature (e.g., heat shock), heavy metal stress (for example, from cadmium), stress produced by a chemical toxicant or small molecule (such as amino acid analogs like azetidine, anti-inflammatory drugs, or arachidonic acid and its derivatives), oxidative stress, oxygen glucose deprivation (OGD), and oxygen deprivation (OD). In certain embodiments, the cell is exposed to an elevated temperature stress. In other embodiments, the cell is exposed to an OGD stress. In certain embodiments, the cell is exposed to endoplasmic reticulum (ER) stress.

In stresses caused by a chemical toxicant, the toxicant may be selected from a protein synthesis inhibitor, proteosome inhibitor, serine protease inhibitor, HSP inhibitor (such as a HSP90 inhibitor), inflammatory mediator, triterpenoid, NSAID, hydroxylamine derivative, flavanoid and another inhibitor of cellular respiration or metabolism. In certain embodiments, the chemical toxicant is rotenone.

Suitable protein synthesis inhibitors include but are not limited to puromycin and azetidine.

Suitable proteosome inhibitors include but are not limited to MG132 and lactacystin.

Suitable serine protease inhibitors include but are not limited to DCIC, TPCK and TLCK.

Suitable inflammatory mediators include but are not limited to cyclopentenone prostaglandins, arachidonate and phospholipase A2.

Suitable triterpenoids include but are not limited to celastrol.

Suitable NSAIDS include but are not limited to sodium salicylate and indomethacin.

Suitable hydroxylamine derivatives include but are not limited to bimoclomol, arimoclomol, and iroxanadine.

Suitable flavanoids include but are not limited to quercetin.

Suitable other inhibitors include but are not limited to benzylidene lactam compounds, e.g., KNK437 and HSP90 inhibitors, e.g., radicicol, geldanamycin and 17-AAg.

In certain embodiments, the cellular stress is an elevated temperature stress (e.g., a temperature above ambient temperature), which comprises elevating the temperature at which the cells are cultured to less than 47° C., such as, less than 45° C., 43° C., or 42° C. For example, the elevated temperature stress may comprise culturing cells at a temperature of from about 35° C., 36° C., 37° C., 38° C. or 39° C. to just below or less than 42° C., 43° C., or 45° C. or to about 42° C., 43° C., or 45° C. In other embodiments, the elevated temperature stress comprises elevating the temperature at which the cells are cultured to from about 39° C. to less than or about 43° C., for example, at a temperature of from about 39° C., 40° C., 41° C., or 42° C. to less than or about 43° C. In some embodiments, the elevated temperature stress comprises elevating the temperature at which the cells are cultured to from about 39° C. to less than or about 42° C., for example, at a temperature of from about 39° C., 40° C., or 41° C. to less than or about 42° C. In other embodiments, the elevated temperature stress comprises elevating the temperature at which the cells are cultured to about 41° C. In other embodiments, the elevated temperature stress comprises elevating the temperature at which the cells are cultured to approximately 41° C., for example, to 41° C.±1.8, 1.5, 1.2, 1.0, 0.8, 0.6, 0.5, 0.4, 0.2 or 0.1° C., such as 41° C.±0.5° C. In further embodiments, the elevated temperature stress comprises elevating the temperature at which the cells are cultured to about 43° C. In other embodiments, the elevated temperature stress comprises elevating the temperature at which the cells are cultured to approximately 43° C., for example, to 43° C.±1.8, 1.5, 1.2, 1.0, 0.8, 0.6, 0.5, 0.4, 0.2 or 0.1° C., such as 43° C.±0.5° C.

In certain embodiments, the method is a high-throughput method for identifying activators of HSP expression or HSF expression. In some of such instances, the cellular stress is an elevated temperature stress, which comprises elevating the temperature at which the cells are cultured to approximately 41° C., for example, to 41° C.±1.8, 1.5, 1.2, 1.0, 0.8, 0.6, 0.5, 0.4, 0.2 or 0.1° C., such as 41° C.±0.5° C. In particular such instances, the method is a high-throughput method for identifying activators of HSF expression, such as HSF1 expression, and/or activators of HSP expression, such as HSP70 expression, and the cells are cultured to about 41° C. or approximately 41° C.±0.5° C.

In certain embodiments, the method is a high-throughput method for identifying inhibitors of HSP expression or HSF expression. In some of such instances, the cellular stress is an elevated temperature stress, which comprises elevating the temperature at which the cells are cultured to approximately 43° C., for example, to 43° C.±1.8, 1.5, 1.2, 1.0, 0.8, 0.6, 0.5, 0.4, 0.2 or 0.1° C., such as 43° C.±0.5° C. In particular such instances, the method is a high-throughput method for identifying inhibitors of HSF expression, such as HSF1 expression, and/or inhibitors of HSP expression, such as HSP70 expression, and the cells are cultured to about 43° C. or approximately 43° C.±0.5° C.

In some instances, the heat shock induced by any of the methods of the present invention may be a mild heat shock. In certain embodiments, cells are treated with a preconditioning, sublethal stress. This preconditioning treatment of cells may allow cells to better tolerate/adapt to lethal stress. The preconditioning stress may be strong enough to reach a submaximal heat shock response.

In certain embodiments, the step of elevated temperature stress is achieved using a thermostat controlled heated plate made from a conducting metal, such as aluminum. This aluminum plate may be custom made to fit the appropriate apparatus used in the experiment and may be heated to maintain the appropriate temperature. This aluminum plate is capable of producing better heat transduction when compared to conventional heating methods. As one of skill in the art will readily appreciate, other metals, solids or semi-solid materials, or even heat-holding liquids, may be used to construct a plate system by which the temperature of multiple cell samples may be controlled precisely. Such materials may be substitutes for the aluminum plate described herein.

As discussed above, the combination of variables measured in the instant methods may be any combination selected from the CV of nuclear intensity, granule count, granule area, granule intensity, and ratio of granule intensity to background intensity. In certain embodiments the combination of variables is the CV of nuclear intensity and granule count. In other embodiments, the combination of variables is the CV of nuclear intensity and granule area. In other embodiments, the combination of variables is granule count and granule area. In other embodiments, the combination of variables is the CV of nuclear intensity and granule intensity. In other embodiments, the combination of variables is the CV of nuclear intensity and the ratio of granule to background intensity. In still other embodiments, the combination of variables is granule count and the ratio of granule intensity to background intensity. In certain embodiments, the imaged granules may be HSP granules. In other embodiments, the imaged granules may be HSF granules, such as HSF1 granules. In yet other embodiments, the imaged granules may be HSP and HSF1 granules. In some instances, a given granule may be homogeneous, e.g., composed substantially of only HSP or HSF. In other instances, a given granule may heterogeneous, e.g. composed of both HSP and HSF and/or additional nuclear matter.

The high-throughput methods of the present invention may be used to measure the modulation of expression of any HSP. Some specific examples of HSPs suitable in the present method include, but are not limited to HSP10, HSP27, HSP60, HSP70, HSP71, HSP72, HSP90, HSP104 and HSP110. In some preferred embodiments, the heat shock protein used in the present method is HSP70.

The high-throughput methods of the present invention may utilize a variety of different types of cells for screening purposes, e.g., cancer cells, neuronal cells, or neuronal cancer cells. The cells used in the high-throughput method may be immortalized cells, primary cells (e.g., fibroblasts and epithelial cells), and/or transformed cells, such as from human transformed cell lines. Suitable non-limiting examples include HOS (hyperdiploid osteosarcoma cell line) and A431 (hypotetraploid epidermal carcinoma cell line).

In certain embodiments, the neuronal cell line is selected from but not limited to an ACN, BE(2)-C, BE(2)-M17, CHP-212, CHP-126, GI-CA-N, GI-LI-N, GI-ME-N, IMR-32, IMR-5, KELLY, LAN-1, LAN-188, LAN-5, MHH-NB-11, NB-100, NGM96, NGP96, SH-SY5Y, SIMA, SJ-N-KP, SK-N-AS, SK-N-BE(2), SK-N-DZ, SK-N-F1, SK-N-MC, SK-N-SH, or Neuro-2a cell line. In certain embodiments, the neuronal cell line is an SH-SY5Y cell line.

In certain embodiments, the cancer cells used in any of the present methods are selected from but not limited to carcinoma cells, sarcoma cells, esophageal cancer cells, etc. Suitable non-limiting examples of cancer cells include a HeLa, A549, DLD-1, DU-145, H1299, HCT-116, HT29, K-562, MCF7, MDA-MB-231, NCI-H146, NCI-H460, NCI-H510, NCI-H69, NCI-H82, OVCAR-3, Paca-2, PANC-1, PC-3, Saos-2, SF-268, SK-BR-3, SK-OV-3, SW-480, SW-620, WM-266-4, HL-60, TE-2, or K-562 cell line. In certain embodiments, the cancer cell line is a HeLa cell line.

In some embodiments, the HCS is automated and makes it possible to screen a large number of compounds with a reasonably fast throughput. In some embodiments, the HCS contains advanced imaging software to significantly improve complicated image segmentation and high speed data processing. In certain embodiments, the HCS can be used to screen different stress conditions. In certain embodiments, the different stress conditions are selected from temperature, OGD, rotenone, and ER induced stress as well as others listed herein.

In some embodiments, the methods for identifying modulators described above can be further combined with one or more known secondary assays to provide modulators with more favorable properties e.g., cytoprotection. In some embodiments, the secondary assay is an MG-132 assay or any of a number of other assays that provide analogous data, such as data on cytoprotective effects. See e.g., Sun F. et al., Neurotoxicology 2006 27 (5): 807; Jullig M, et al., Apoptosis 2006 11 (4): 627; and Valenta E M, et al., Science 2004 304: 1158.

In some embodiments, the secondary assay is an MTS assay or any a number of other assays that provide analogous data, such as data on cytotoxic effects. The skilled artisan would understand that the MTS assay can be used to identify compounds with cytotoxic effects. In certain embodiments, the methods for identifying modulators described above can be combined with both an MG-132 assay and a MTS assay.

### Modulating the Level of Baseline Stress in Cells

In certain embodiments, a measuring step in the high-throughput method comprises measuring the level of HSP and/or HSF expression in a cell treated with a selected type of cellular stress and comparing it to the baseline level of HSP and/or HSF expression in a cell not treated with the stress to quantitatively measure the change in HSP and/or HSF expression associated with the stress treatment. In certain embodiments, the HSP and/or HSF expression associated with the stress treatment is an increase in HSP and/or HSF expression over the baseline level of expression. In other embodiments, the HSP and/or HSF expression associated with the stress treatment is a decrease in HSP and/or HSF expression below the baseline level of expression.

Accordingly, in certain embodiments, the baseline level of HSF and/or HSP expression in cells to be treated with putative modulators may itself be externally modulated or selected, for example by pretreatment with a known HSF and/or HSP modulator (either activator or inhibitor), so that relatively small changes in expression in either direction may be detected upon treatment with putative modulators of the cellular stress response according to the methods of the invention. The step of externally modulating or selecting a baseline level of HSF and/or HSP expression is optionally performed to fine tune, for each cell type or each type of modulator to be tested, the sensitivity of the assay, e.g., to increase the signal to noise ratio and ultimate sensitivity of the assay. Thus, in certain embodiments of the invention, the baseline of HSP and/or HSF expression may be externally altered to increase the sensitivity of the assay, so that changes in expression in either direction may be more accurately or more readily detected.

Selection of a low or intermediate level of cellular stress to be applied to a given cell type, for example, may enable up or down modulators to be identified using the methods of the invention that would otherwise have been missed by performing the same steps on cells tested at a higher (or lower) level of cellular stress. Modulation of the baseline level of cellular stress for use in identifying modulators of heat shock response in the assays of the invention may be accomplished by performing dose and time response curves for a given cell type treated with one or more selected stresses and determining an optimal range of time and dose of stress treatment to achieve increased or optimal sensitivity in the ability to select modulators (activators or inhibitors) of HSF and/or HSP expression.

### Devices

In certain embodiments, the elevated temperature of the heat shock methods described herein is applied and maintained by a heating apparatus comprising a plate, such as a metal plate, such as an aluminum plate. The plate may be custom made to fit the appropriate apparatus used in the experiment and may be uniformly heated to maintain the appropriate temperature. The plate is capable of producing better heat transduction when compared to conventional heating methods, such as a water bath, thereby resulting in consistent and accurate heat shock of cellular samples.

Hence, in certain instances the present invention includes a device for inducing heat shock stress in a plurality of cellular samples, the device comprising a plate and a heating source for heating the plate, wherein the plate is positioned so as to transfer heat uniformly to the plurality of cellular samples. In some instances, the plate is a metal plate, such as steel, copper, or aluminum plate, particularly an aluminum plate, or an alloy, for example comprising steel, copper, or aluminum. In other instances, the plate is a glass or other non-metal plate. In certain embodiments, the plate directly contacts each of the cellular samples in the plurality of cellular samples. The plate may promote uniform transfer of heat from the heating source to each of the samples, thereby inducing uniform heat shock in each of the samples. For example, the plate may be used in conjunction with a multi-welled plate (e.g., a 96-well plate or greater) containing a plurality of samples. In certain embodiments, the plate may be directly associated, e.g. directly contacting, the multi-welled plate. For example, the multi-welled plate may rest on top of the plate.

Modulators for Diagnostic and/or Therapeutic Treatment Methods

In certain embodiments, modulators identified by the high-throughput methods of the invention will be useful in diagnostic methods relating to a disease, condition or indication accompanied by a physiological stress which has a cellular stress response component. Diagnostic methods and kits for performing the diagnostic methods are provided.

Modulators of HSF and/or HSP identified according to the present invention (and derivatives thereof in which the modulator is linked to a heterologous moiety such as a radioactive, fluorescent, phosphorescent, nucleic acid, antibody, or protein-based tag) may be useful tools for diagnosing the cellular stress state of a cell or cell population. In addition, nucleic acid molecules encoding a modulator of the invention (or nucleic acid molecules which can bind to nucleic acid regulatory regions of other nucleic acid molecules which encode a modulator of the invention) may be designed to express, detect, and/or to regulate expression of the modulator in a cell. Vectors comprising said nucleic acid molecules, and cells comprising said nucleic acid molecules or vectors of the invention are also provided.

In other embodiments, modulators identified by the high-throughput methods of the invention will be useful in methods to treat or prevent a disease, condition or indication accompanying a physiological stress which has a cellular stress response component. In other embodiments, the modulators identified by the high-throughput method are used to produce a medicament to treat or prevent a disease, condition or indication accompanying a physiological stress which has a cellular stress response component. The disease or indication may be in a human or in a non-human animal.

In some embodiments, the disease, condition or indication is selected from a cardiovascular disease, vascular disease, cerebral disease, allergic disease, immune disease, autoimmune disease, a viral or bacterial infection, skin disease, mucosal disease, epithelial disease, or a disease of the renal tubuli, for example.

In certain embodiments, the cardiovascular disease is atherosclerosis, coronarial disease, or cardiovascular disease caused by hypertonia and pulmonary hypertonia.

In certain embodiments, the cerebral disease is cerebrovascular ischemia, stroke, traumatic head injury, senile neurodegenerative disease such as senile dementia, AIDS dementia, alcohol dementia, Alzheimer's disease, Parkinson disease or epilepsy.

In certain embodiments, the skin or mucosal disease is a dermatological disease or an ulcerous disease of the gastrointestinal system.

In certain other embodiments, especially in embodiments in which HSF1/HSP modulators are inhibitory, the disease, condition or indication involves general, regulated and/or targeted cell cytotoxicity, apoptosis or other types of cell death. Treatment of any of a number of cancers, tumors or other cells or cell types that exhibit abnormal growth or cell division, e.g., have which have lost normal growth control, including virally infected cells, are included.

### Exemplification

With aspects of the claimed invention now being generally described, these will be more readily understood by reference to the following examples, which are included merely for purposes of illustration of certain features and embodiments of the presently claimed invention and are not intended to be limiting.

### Master Chaperone Regulator Assay (“MaCRA”)

The “Master Chaperone Regulator Assay” or “MaCRA” was developed as a high content, cell image based assay for identifying modulators of stress response pathways in cells, such as HSF1 and HSP70. The MaCRA assay has been developed into a high throughput screening method which is capable of identifying different classes of cell stress response modulatory compounds based on their performance in each of multiple assays (see below). The development of the MaCRA assay is described below as an example of how one may design a series of assays looking at different parameters of the shock response pathway in cells. Example 1 describes the development of a high content, image based HSF/HSP granule assay in screening and EC50 formats designed to identify HSF1 activators, as described in more detail below.

### Optimization of Heat Shock Screening Conditions for HCS Granule Assay

In the HCS setting, a known HSF1 activator celastrol (Westerheide S D, et al. J. Biol. Chem. 2004; 279(53):56053-60) was used as the assay positive control. Notably, celastrol can induce HSF1 activity in both stressed and non-stressed cells. Thus, celastrol does not meet a preset definition of a compound characterized as a HSF1 amplifier.

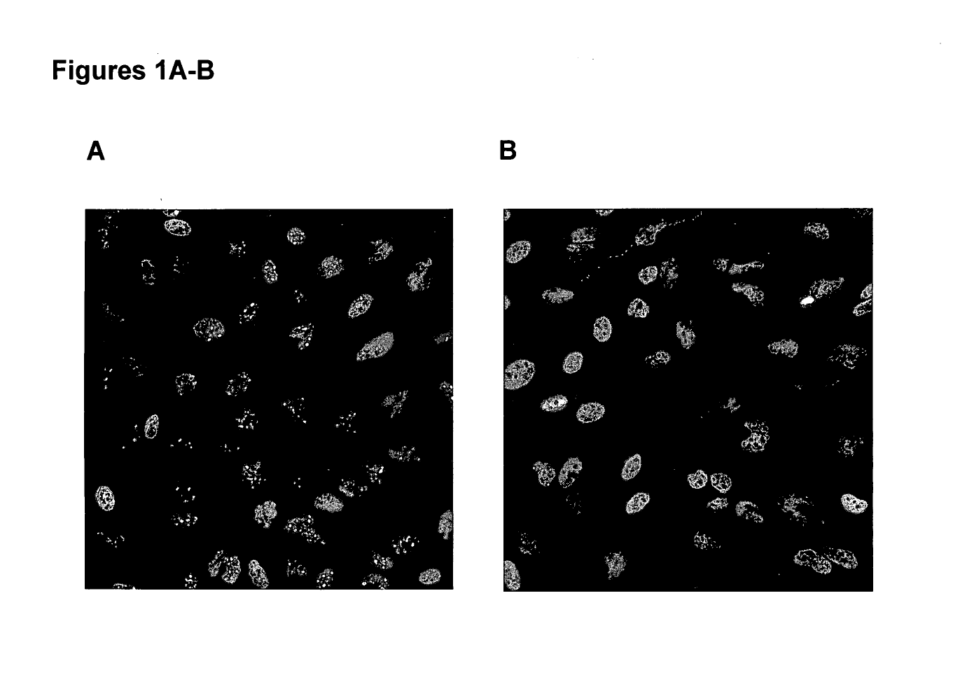

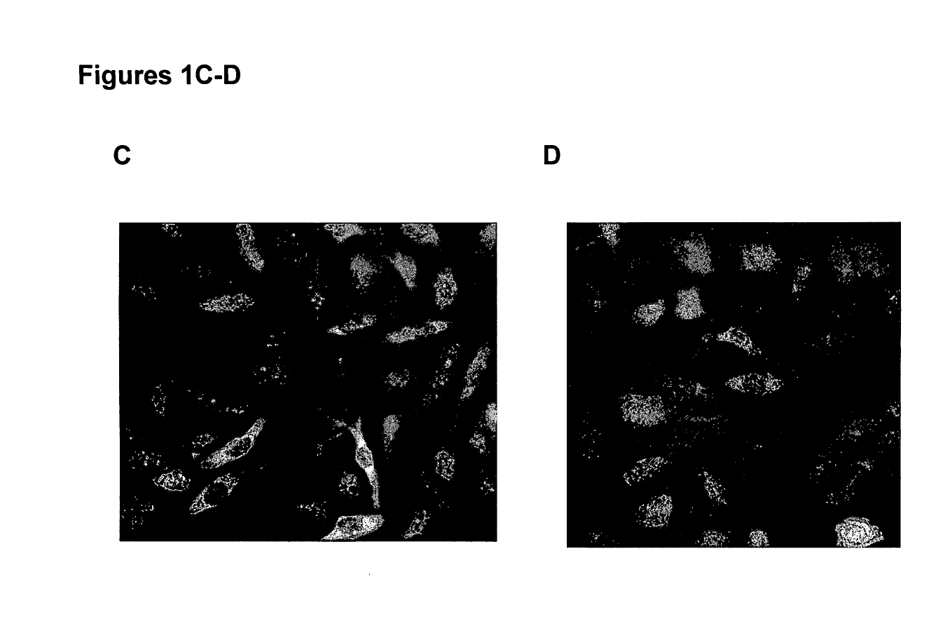

For heat shock based screening, two technical challenges had to be overcome. First, the temperature and time of heat shock had to be optimized. Different heat shock temperatures and recovery times have been reported in the literature (Cotto J. et al., J Cell Sci. 1997; 110 (Pt 23):2925-34; Westerheide et al., supra). Higher temperature (such as 43° C. or greater) leads to a significantly high background in DMSO treated samples, diminishing the signal/noise ratio of compound amplification effects. The optimized condition was selected empirically to be at approximately 41° C. for 2 hours with no recovery time, under which a satisfactory window was observed for HSF1/HSP70 granule formation induced by 2 μM celastrol (FIGS. 1A and C) as compared to DMSO solvent control (FIGS. 1B and D).

Second, in general, it is difficult to ensure that heat is evenly transduced to each well when heat shock experiment is performed in a 96-well or higher well plate format. When regular air heat (from incubator) was used for heat shock experiment, a large variability (CV>40%) of HSF1/HSP70 granule count throughout the 96-well plate was observed. A high throughput heat shock method was reported previously to immerse the plates in a 45° C. water bath, which is not suitable for image based high content method (Zaarur et al., Cancer Res. 2006; 66(3):1783-91). One solution was to use a custom made aluminum plate for quick heat transduction, which enabled performance of 96-well plate based heat shock with a relatively low CV (7.94%) for HSF1 granule count.

### Quantification of HSF1/HSP Stress Granules and Assay Validation

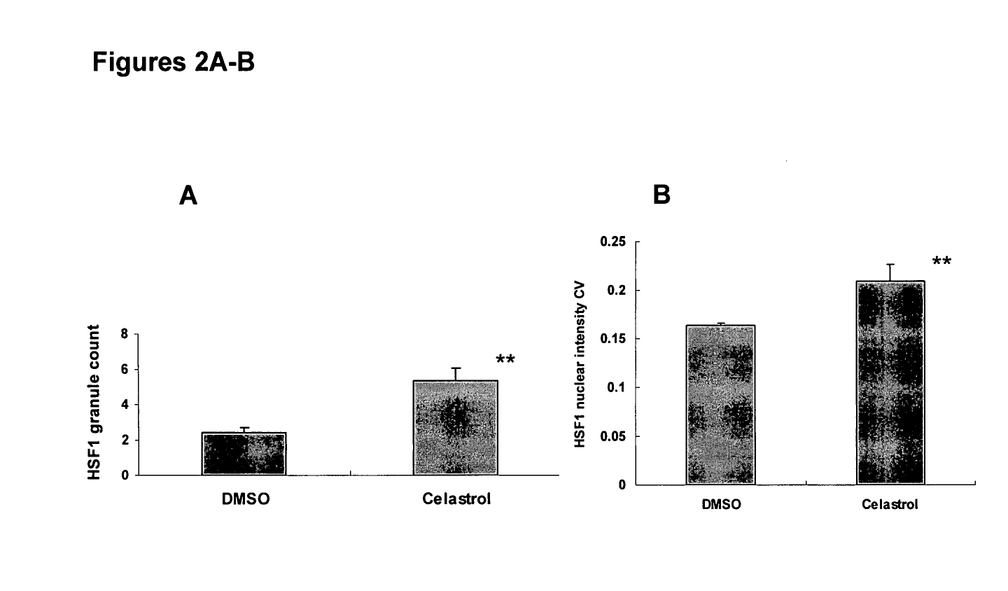

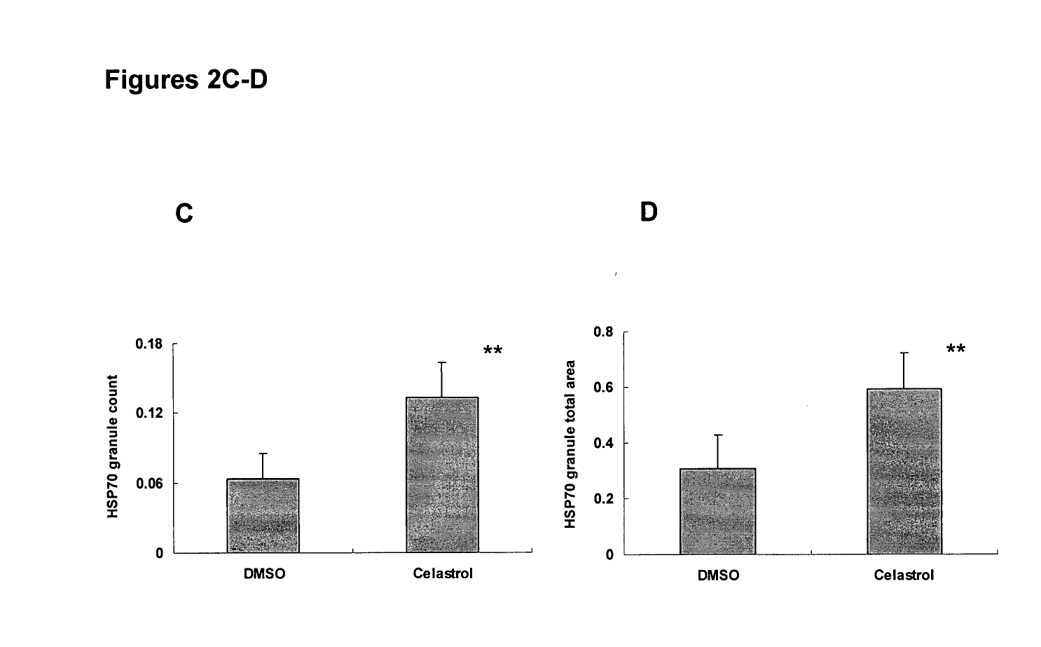

Next, several image parameters of the observed granulate particles were quantitated. The Multi Target Analysis (MTA) module from Workstation software (GE Healthcare) provides a high-speed measurement of nuclear granules including granule count, granule area, granule intensity and nuclear intensity CV (which measures CV of pixel intensity in the nucleus). As shown in FIGS. 2A-D, granule count and nuclear intensity CV was chosen for quantification of HSF1 granules (FIGS. 2A and 2B) while granule count and granule area were adopted for measuring signal of HSP70 granules (FIGS. 2C and 2D). The data in FIG. 2A indicates that heat shock (41° C. for two hours) induced an average 5.34±0.72 HSF1 stress granules per nucleus in HeLa cells exposed to 2 μM of celastrol as quantified by MTA. By comparison, DMSO-treated cells contained 2.46±0.22 granules. In order to achieve a relatively low background, 5 was selected as the threshold for HSF1 granule count induced by compound treatment. HeLa cells that contained more than 5 HSF1 granules were designated as “HSF1 granule positive cells.” The thresholds for HSF1 nuclear intensity CV, HSP70 granule count and HSP70 granule area were also chosen with gating values equivalent to the average of DMSO treated samples plus 2 or more standard deviations (FIGS. 2B-2D). These results show a statistically significant increase in the number of HSF1 and/or HSP70 positive granules (granule count; FIGS. 2A and C), in the intensity of HSF1 positive granules in the nucleus (FIG. 2B) and in the total area of HSP70 positive granules (FIG. 2D) in celastrol-treated versus control (DMSO) treated HeLa cells hence validating the use of these four parameters, alone and preferably in various combinations, for quantification of HSF1 and HSP70 signals, and modulation thereof by test compounds or conditions.

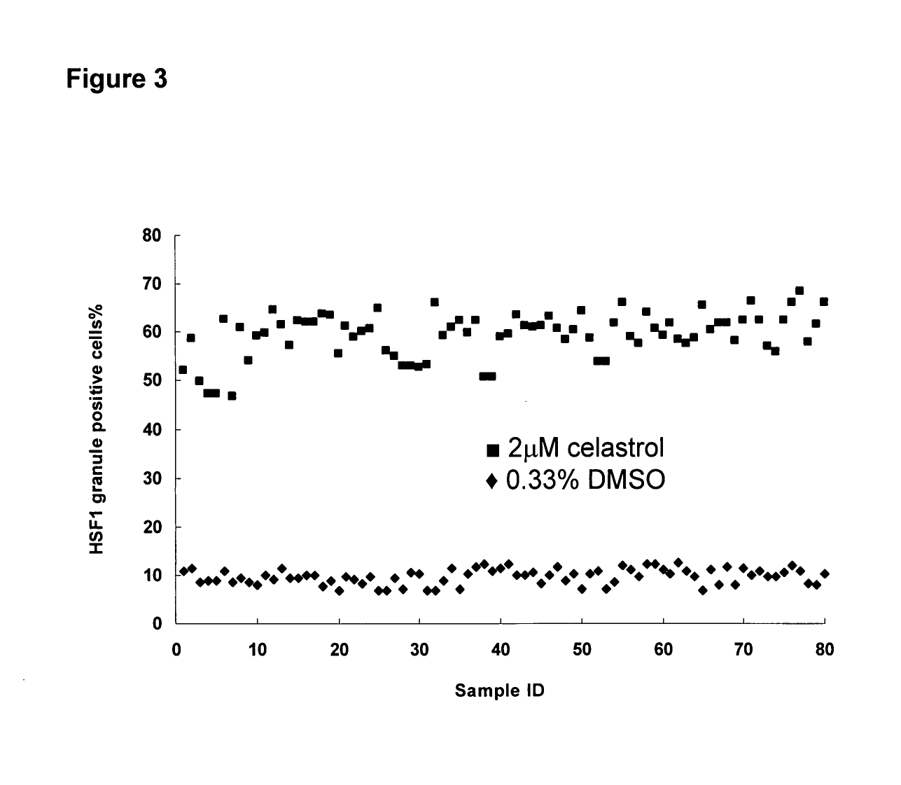

High content screening (HCS) granule assay performance in the 96-well plate format was also evaluated with samples treated with 2 μM of celastrol one hour prior to heat shock as positive controls and samples treated with 0.33% DMSO as negative controls. As shown in FIG. 3, the experimental data show that the HSF1 granule assay produces a wide screening window and Z′. (The Z-factor is a measure of the quality or power of a high-throughput screening (HTS) assay; Z-factor analysis was performed as described in Zhang et al., J. Biomol. Screen. 2008; 13(6):538-43).

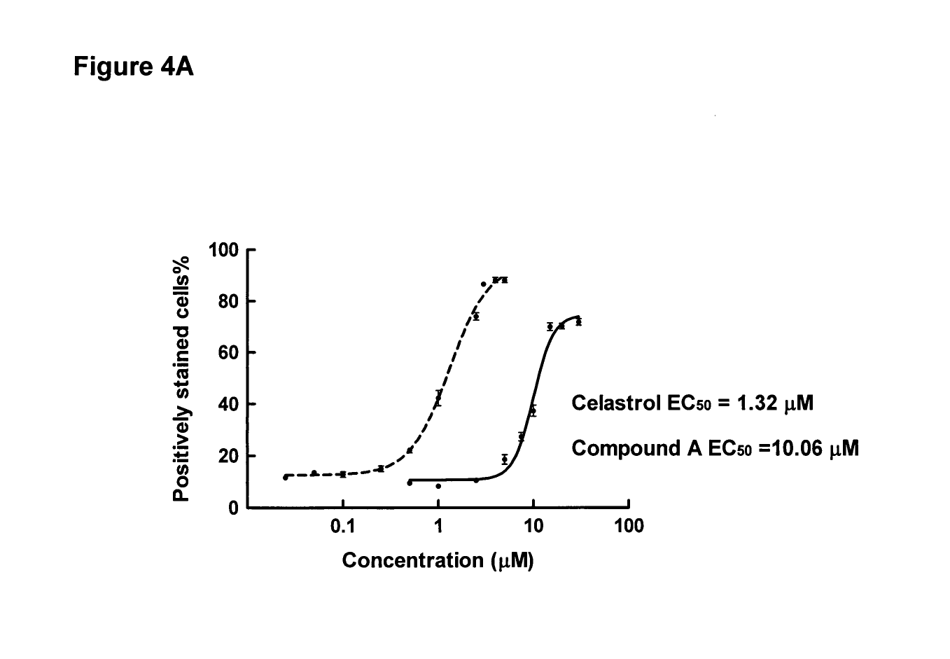

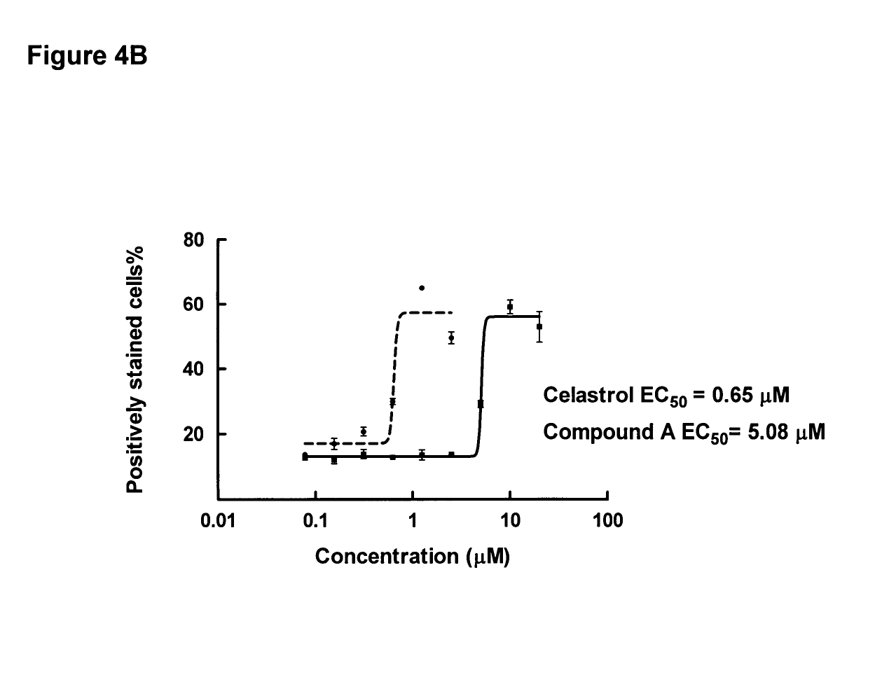

Several positive hits from the initial high throughput screen were selected to examine their dose dependency for HSF1 coinduction, as further described in Example 1. As an example, FIG. 4A shows a dose dependent study with EC₅₀ values of HSF1 intensity CV for celastrol (positive control) and Compound A (a new modulator identified by the present HCS method). The positive control celastrol exhibited an EC₅₀ value of 1.32 μM. This result agrees well with the previously published EC₅₀ value of 3 μM in HeLa cells when HSP70.1 promoter-luciferase reporter was used for characterization of celastrol activated heat shock response. See Westerheide et al., J. Biol. Chem., 279(53):56053-56060 (2004). Similarly, FIG. 4B shows a dose dependent study with EC₅₀ values of HSP granule area for celastrol (positive control) and Compound A. The positive control celastrol exhibited an EC₅₀ value of 0.65 μM. Although Compound A and other hits are not as potent as celastrol, these hits represent good starting points for structure-activity based studies. Notably, unlike celastrol, Compound A does not stimulate or induce HSF1/HSP70 granule formation in normal cells that are not treated with heat shock stress, suggesting that it (or a derivative compound) is a candidate agent that mediates chaperone amplification.

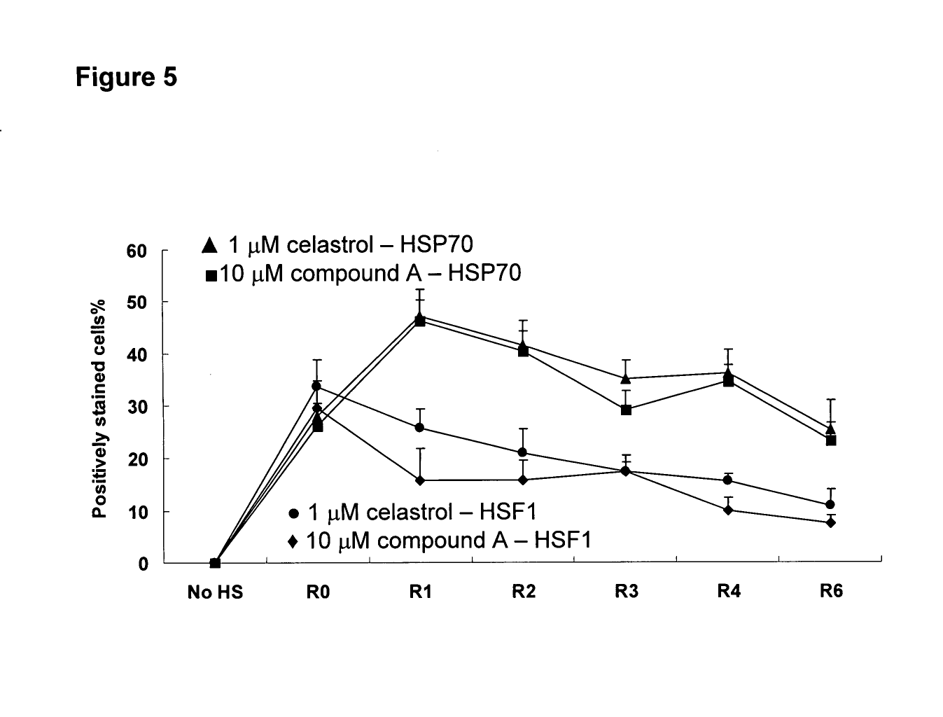

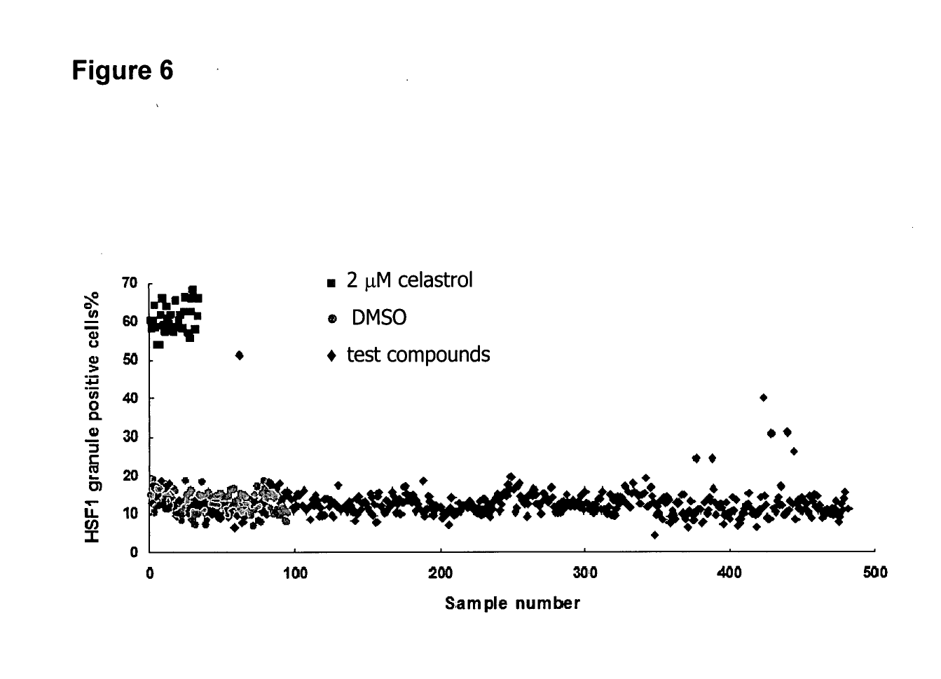

To compare the kinetics of Compound A and celastrol on HSF1/HSP70 induction, a detailed time course study (up to 6 hours in the recovery time) was performed, as further described in Example 1 (see also FIG. 5). The concentrations used in this study were 1 μM and 10 μM for celastrol and Compound A, respectively, which are close to their EC₅₀ values. FIG. 5 shows a comparison of HSF1 induction kinetics in cells over a 6 hour recovery period (R1-R6) after pretreatment with 10 μM Compound A or 1 μM of celastrol (positive control). Compound A exhibited induction behavior similar to celastrol at most time points tested. Both compounds activated HSF1 stress granules for up to six hours after heat shock, strongly suggesting that they may maintain or stabilize the active conformation of HSF1 for continuous induction of HSPs. HSP70 signals peaked one hour after the heat shock and maintained a relatively high level (˜25% positively stained cells) even after 6 hours of the heat shock. At one hour after heat shock, about 30-40% of cells are HSF1+HSP70+. The rest of the cells are HSF1−HSP70+ (˜30%), HSF1+HSP-(˜30%) and HSF1−HSP− (˜10%). The HSP70 signal maintains a relatively high level six hours after completion of heat shock. The long-lasting expression of HSP70 provides an extended protection window for restoring misfolded proteins. FIG. 6 shows data from experiments in which HeLa cells were treated individually with 480 different test compounds (♦) for 30 minutes before a 41° C. heat shock for 2 h with no recovery time. Parallel control treatments were performed using 2 μM celastrol as positive control (▪) and DMSO as negative control (). A number of test compounds (♦) caused a percent (%) increase in HSF1 granule positive cells of at least about 20% (compare to DMSO treated cells which scored below the 20% increase mark). This experiment shows that the assay conditions used in this experiment were sensitive enough to identify from a large set of test compounds a select handful of HFS1 modulatory compounds (here, activators) and confirms the utility of this method for high-throughput screening of such modulatory compounds.

The next question was how to test the cytoprotective effects in a biologically relevant model system. Oxygen glucose deprivation (OGD) is an in vitro system of ischemia and stroke, particularly suitable for neuronal injury studies. Cytotoxicity induced by OGD is primarily due to protein misfolding and aggregation. Overexpression of HSP70 in hippocampal CA1 neurons reduces protein aggregation while neuronal survival is significantly increased. See Giffard et al., J. Exp. Biol., 207(Part 18):3213-3220 (2004); Sun et al., J. Cereb. Blood Flow Metab., 26(7):937-950 (2006). In addition to OGD, the rotenone model of Parkinson's disease is another in vitro system for study of protein aggregation induced cytotoxicity. It has been reported that mitochondria inhibitor rotenone can significantly increase α-synuclein expression which eventually becomes cytoplasmic inclusions similar to Lewy bodies. See Greenamyre et al., Parkinsonism Relat. Disord., Suppl 2:S59-S64 (2003). Therefore, whether these two in vitro cell assay systems could be adapted to evaluate cytoprotective effects of the HCS screening hits was investigated. MTS colorimetric assay reported previously served as the secondary method to measure live cells after various stresses.

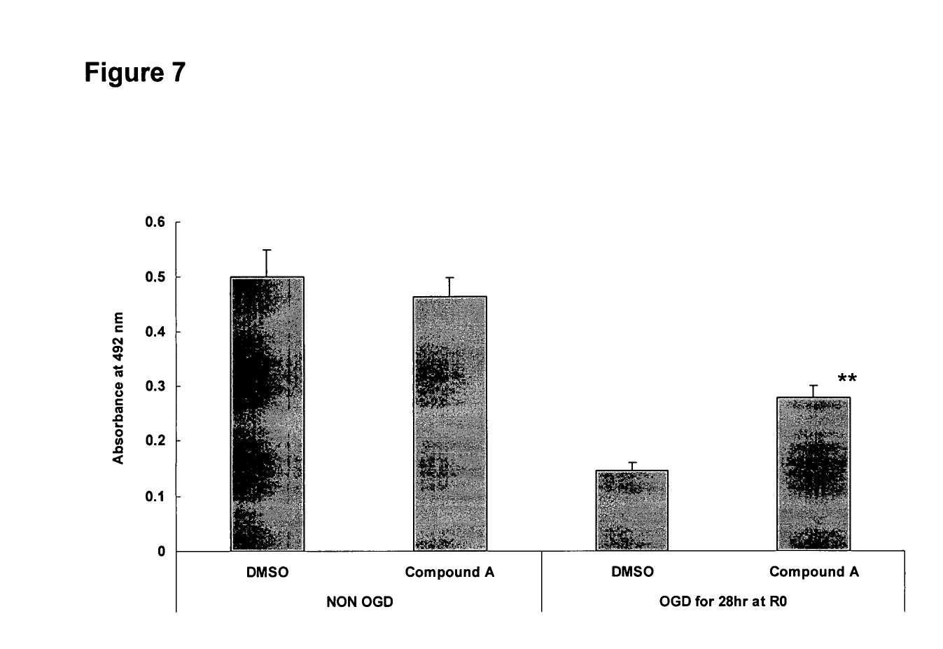

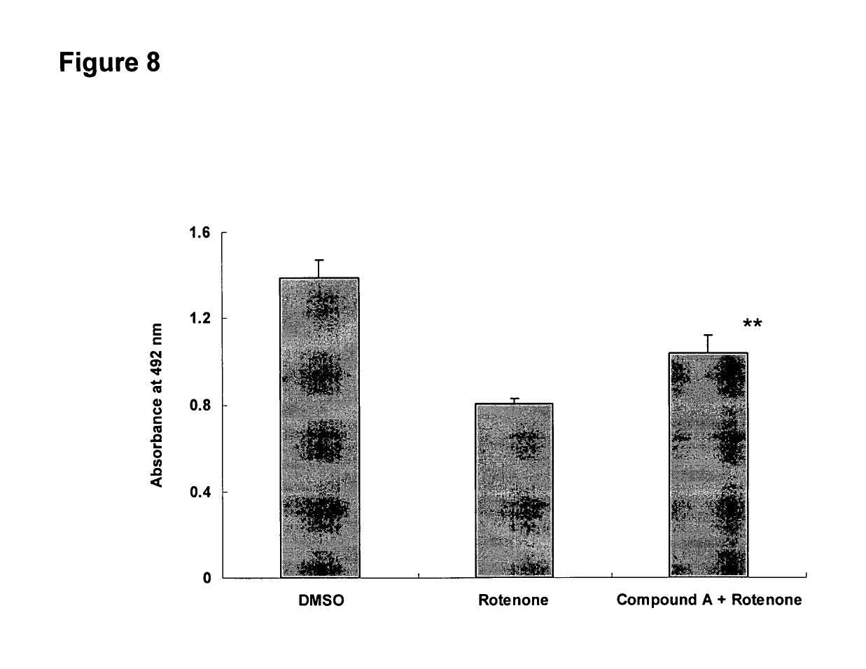

The OGD assay was performed as described in Example 2. As shown in FIG. 7, a 91% increase of viable SH-SY5Y cells was observed when pretreated with 2.5 μM of Compound A, exhibiting significant cytoprotective effects. Notably, there was almost no difference in observed cell viability when SH-SY5Y cells were treated with Compound A and DMSO under normal (no stress) conditions. As for the rotenone model, more than 42% of SH-SY5Y cells were killed when 100 nM of rotenone was applied for 24 hours (FIG. 8). SH-SY5Y cells pretreated with 2.5 μM Compound A resulted in a 29% increase in cell viability compared to DMSO control treated cells (FIG. 8).

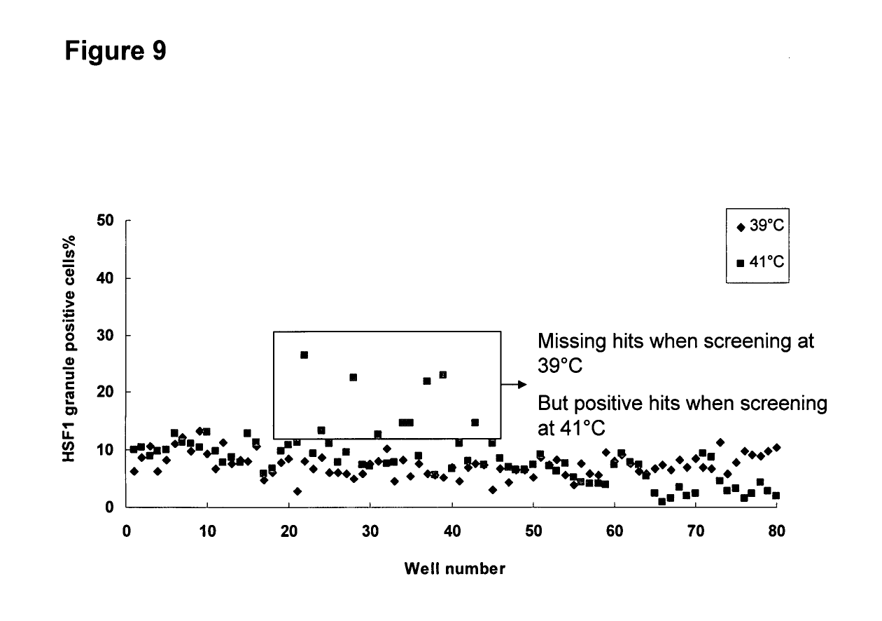

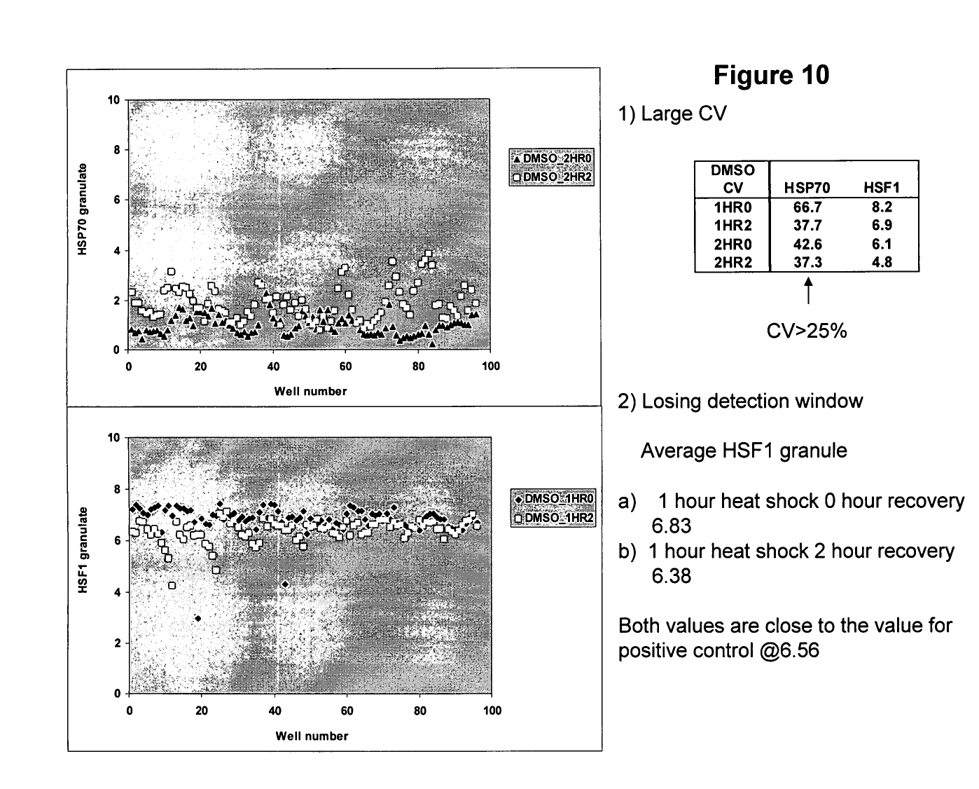

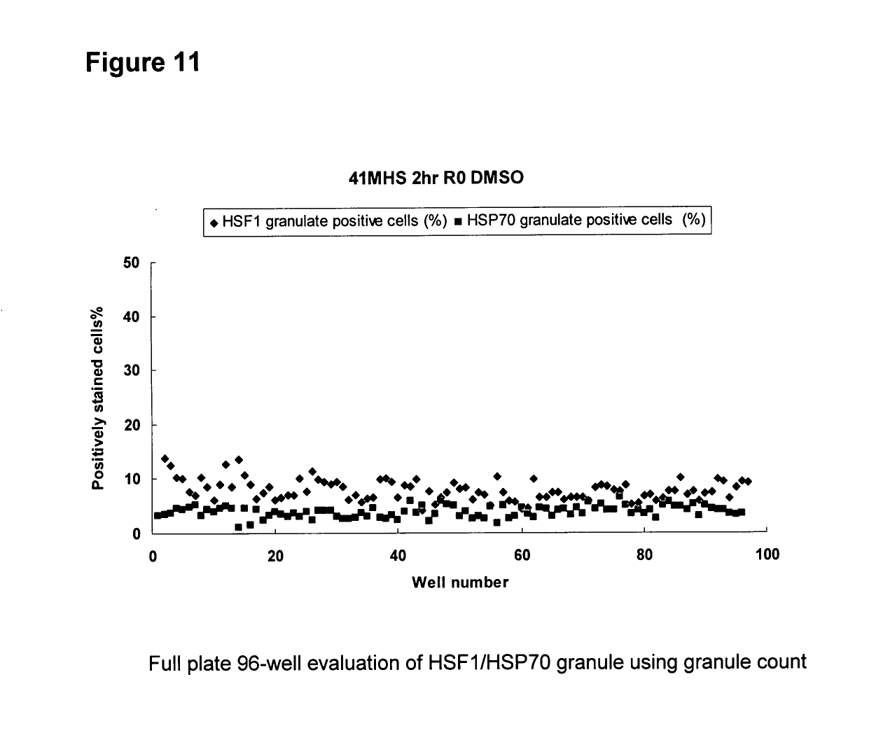

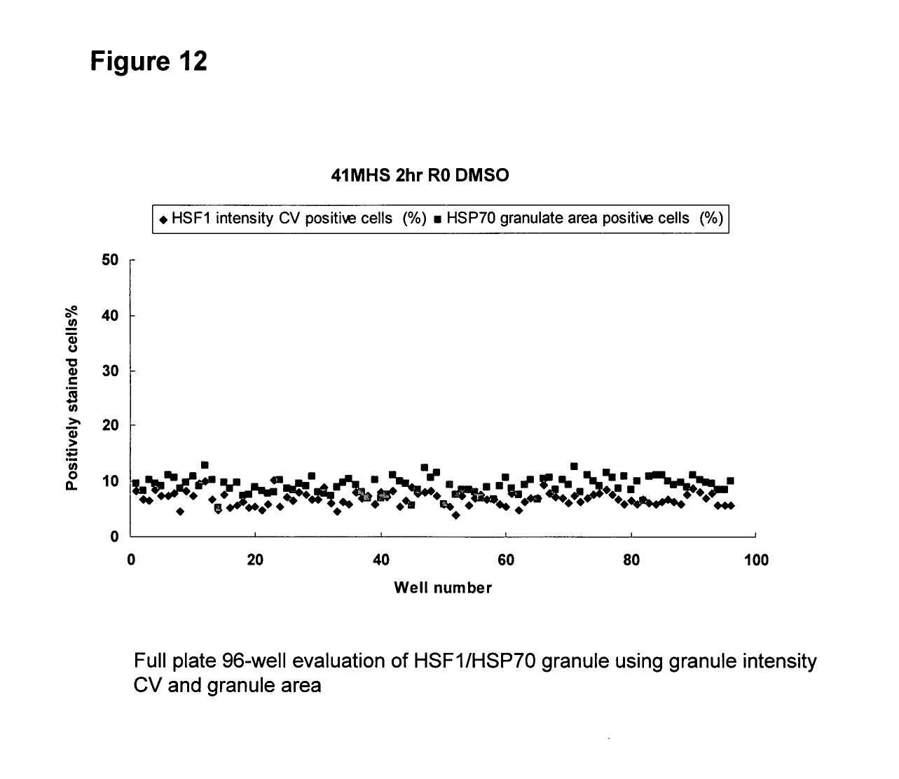

A series of experiments to further optimize assay parameters, such as temperature of heat shock and time of recovery were performed (see Example 4). HSF1 granule positive cells were quantitated in cells treated with stress by elevated temperature at either 39° C. (♦) or 41° C. (▪) for 2 hours with no recovery time (FIG. 9). As shown in FIG. 9, a significant number of positive hits were detected when heat shock was performed at 41° C. for 2 hours with no recovery time compared to heat shock at 39° C. (See (▪) hits at and above the 15% positive cell cutoff.) The data in FIG. 10 show that, for the four heat shock conditions tested at 43° C. (1 hour with 0 hour recovery time, 1 hour with 2 hours recovery time, 2 hour with 0 hour recovery time, and 2 hours with 2 hours recovery time), HSP70 granules in DMSO treated samples are varied, with CV values differing by more than 25% which is unsatisfactory for accurate quantification. The data in FIG. 10 also show that the observed HSF1 positive granule count in DMSO treated samples (negative control) is close to that of celastrol treated samples (positive control) under all tested conditions. Further, the data show that heat shock (43° C. for 1 hour) induced an average of 6.83 HSF1 stress granules per nucleus with no recovery time compared to 6.38 HSF1 stress granules per nucleus with 2 hour recovery time. By comparison, positive controls produce a value of about 6.56, and hence the detection window was less than desirable. FIG. 11 shows HSF1 and HSP70 granule count evaluation for DMSO-treated cells exposed to a 41° C. elevated temperature stress for 2 hours with no recovery period. FIG. 12 shows a HSF1 CV nuclear intensity and HSP70 granule area evaluation for DMSO treated cells exposed to a 41° C. elevated temperature stress for 2 hours with no recovery period. Table 1 summarizes the data in tabular form, showing the CV values for HSF1 granule variable, HSP70 granule variable, HSF1 intensity CV variable and HSP70 granule area when cells are exposed to a 41° C. elevated temperature stress for 2 hours with no recovery period.

TABLE 1

| Heat shock at 41° C. | CV values |
|---|---|
| HSF1 granule | 24.26 |
| HSP70 granule | 25.19 |
| HSF1 intensity CV | 6.9 |
| HSP70 granule area | 9.28 |

The MaCRA HCS granule assays reported herein enable the direct identification of novel chemical entities that can modulate (increase or decrease) HSF1/HSP70 expression under various stress conditions. Compared with conventional Western blot or immunofluorescence assays, the HCS granule assay has at least the following advantages:

1) HSF1/HSP70 stress granules are well correlated with HSF1 and HSP70 activation. Quantification of HSF1/HSP70 granules can measure cellular kinetics of HSF1/HSP70 activity in response to various stressors. Moreover, an improved signal/background was obtained with a better window for compound screening when mild stress condition (heat shock at 41° C.) was applied. This setting also allowed identification of hits with weak induction activity, which were also observed in the high throughput method.

2) Advanced software systems used in conjunction with HCS create a significantly improved platform for complicated image segmentation, cell sorting and analysis, calculation of granule count/area, high speed data processing, etc. In addition, HCS also offers many other phenotypic parameters for compound evaluation. For example, comparison is possible of different compound induced nuclear phenotypic changes (DAPI staining) in the presence or absence of cellular stress, such as heat shock treatment, which can be particularly helpful for prediction of cytotoxicity.

3) The automatic features of HCS enable screening of large compound libraries at higher levels of throughput.

4) The multiplexing nature of HCS assays can be particularly useful in teasing apart complicated biological pathways, which provides a valuable tool to rapidly identify potential targets and biomarkers. A major technical hurdle in heat shock based HCS is to further regulate heat shock operations in a higher (384-well or above) throughput format. To this end, a streamlined and accelerated data processing capacity was developed which has enabled HCS assays described herein to be performed in at least a 384-well format (see below).

### Cytoprotection and Cytotoxicity—Secondary Assays

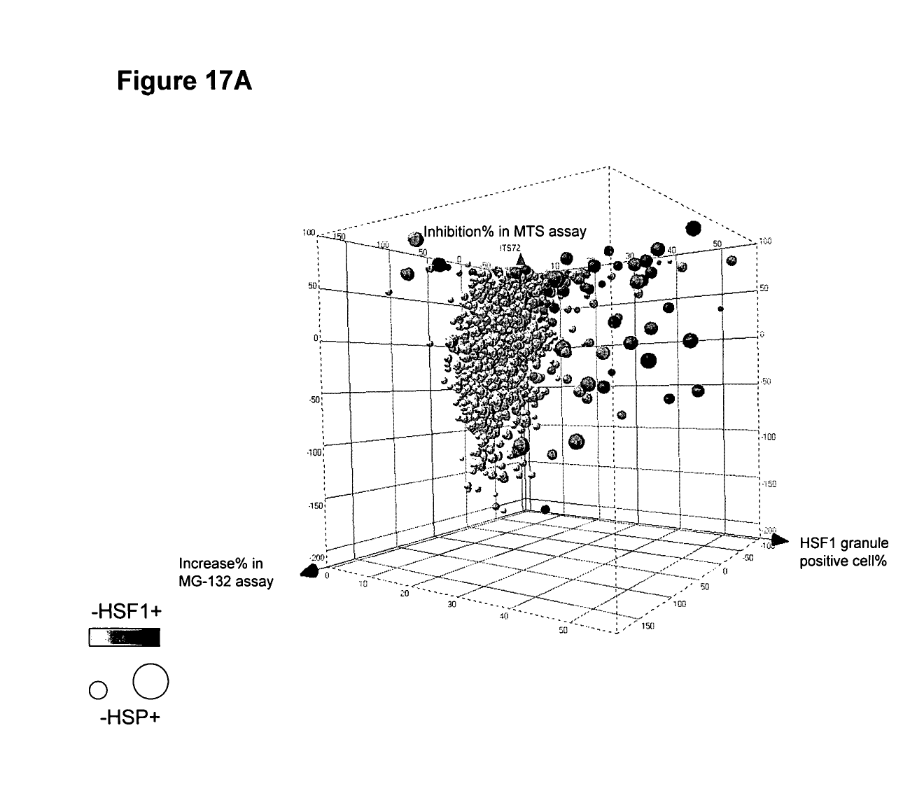

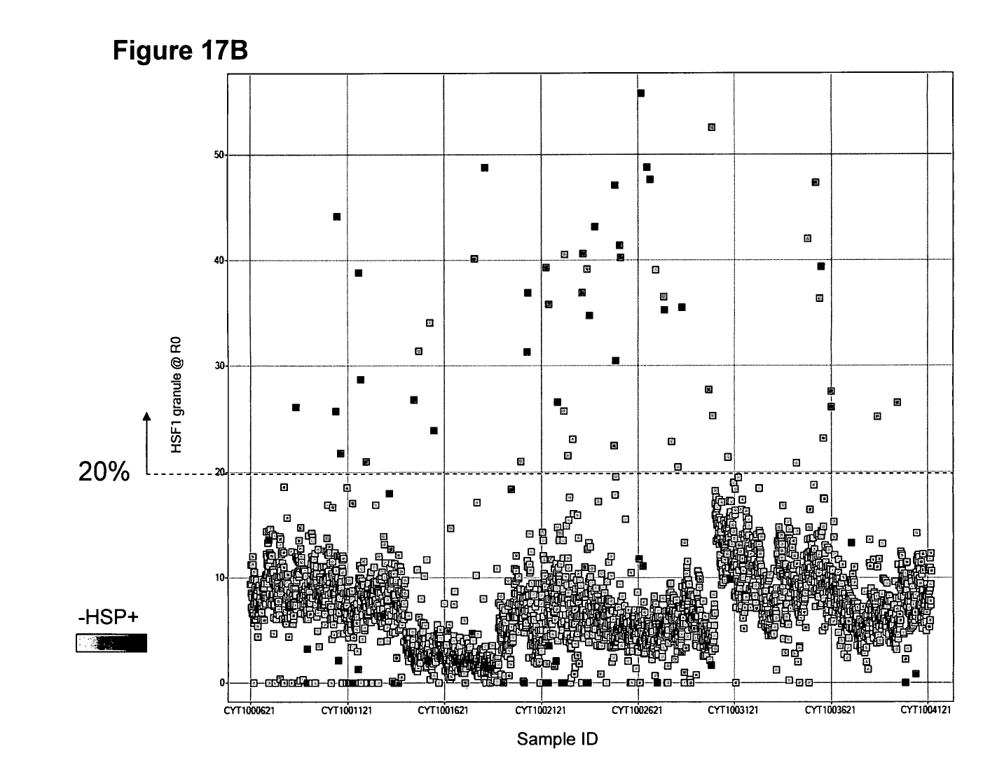

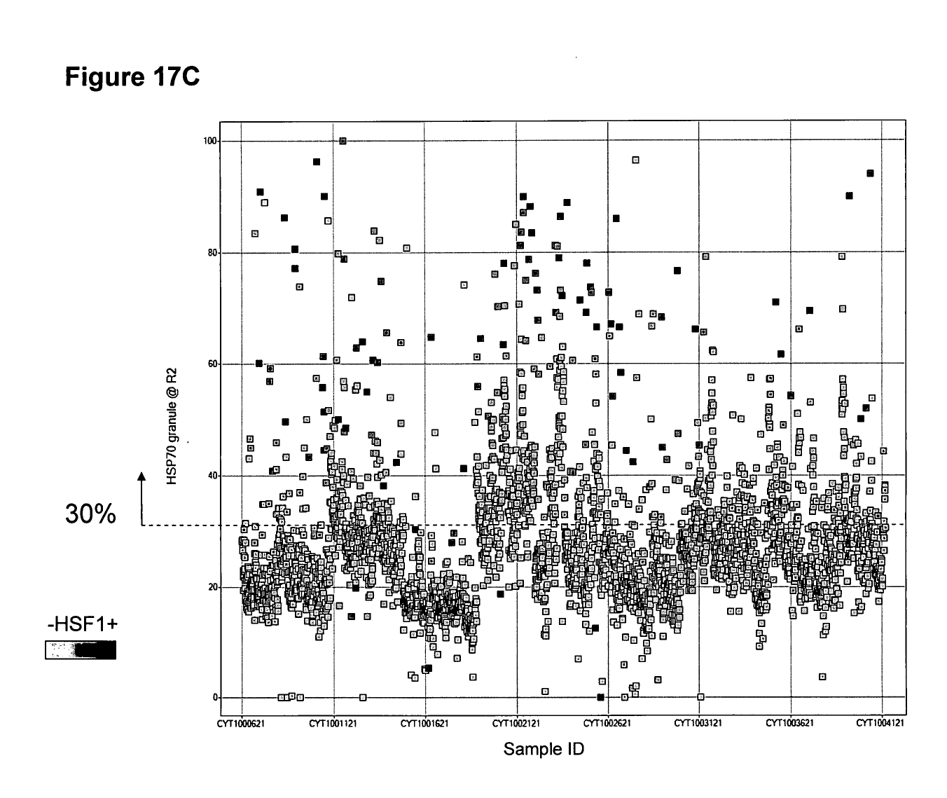

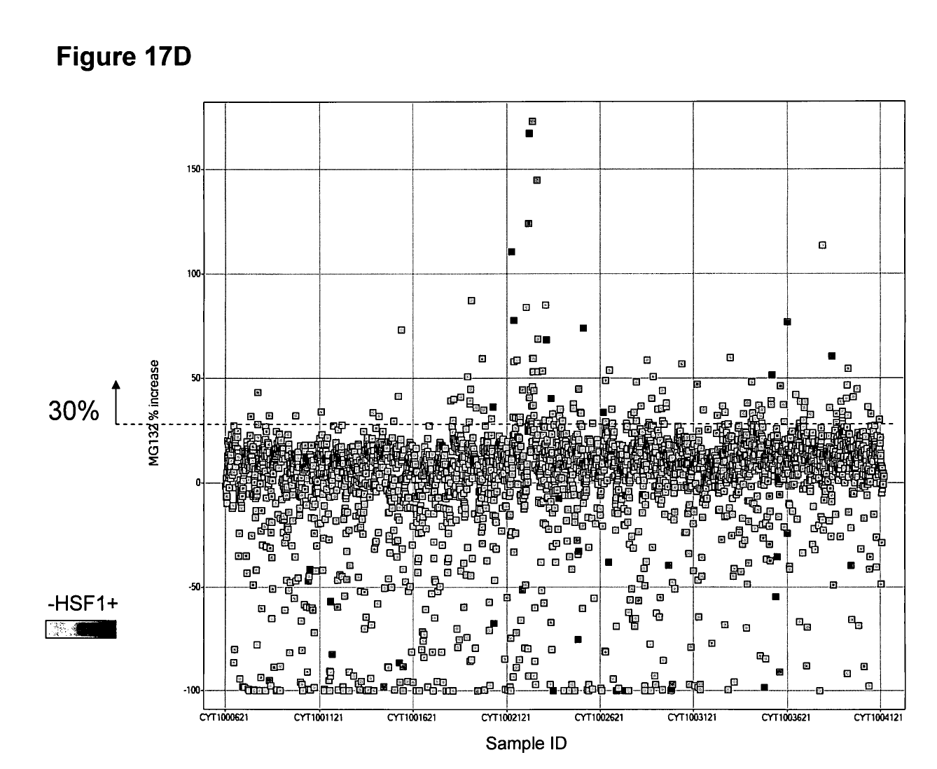

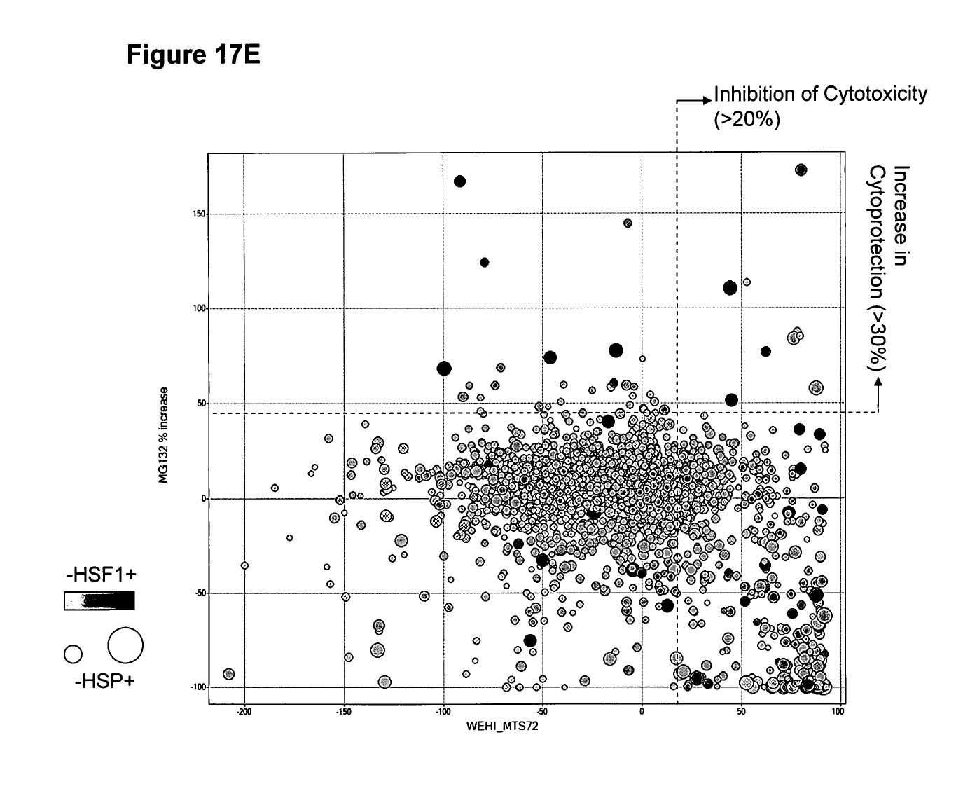

An MTS cytotoxicity assay was used in one or more secondary assays to determine whether the effects seen in HSP70 induction can be translated to cytoprotection. The assay was performed essentially as previously described (Zhang et al., J. Biomol. Screen. 2008; 13(6):538-43; see Example 5). The MTS assay may be used as a secondary assay to determine whether modulators identified in the HSF1/HSP70 screening induce cytotoxicity in cells. (See also FIGS. 17A and 17E, described below.)

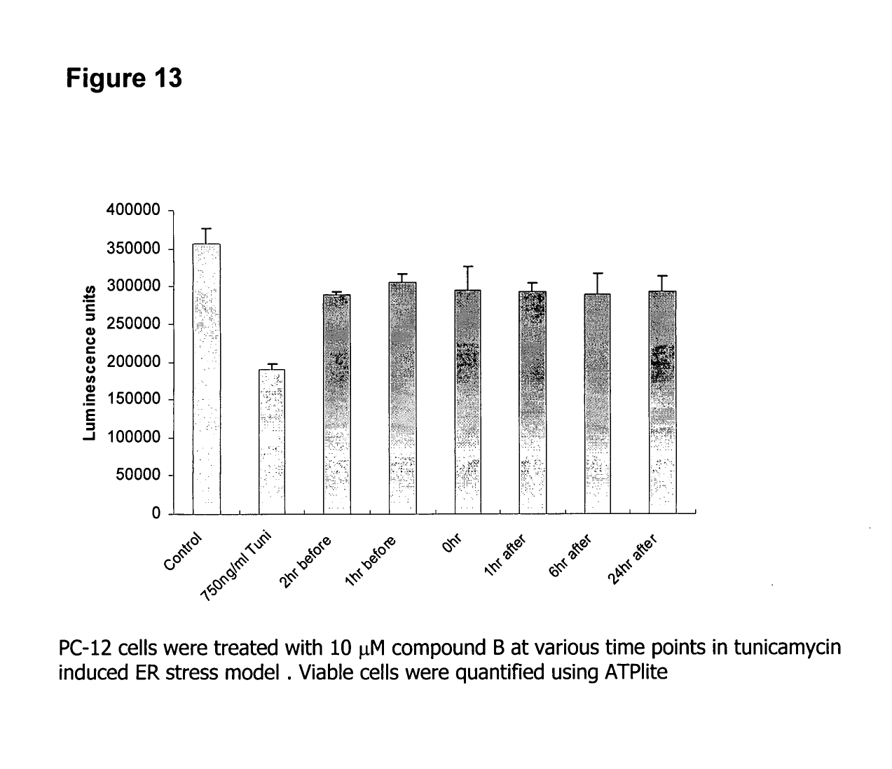

Whether compound hits from MaCRA HSF1/HSP70 granule assay screens are capable of protecting cells from stress to the endoplasmic reticulum (ER) system induced by tunicamycin treatment was tested. As shown in FIG. 13, Compound B at final concentration of 10 μM was added to PC12 cell cultures at various time points in the tunicamycin induced ER stress assay and viable cells were measured as described in Example 6. The data show that cells were significantly protected from tunicamycin induced stress by Compound B treatment before or during tunicamycin treatment, and even out to 24 hours after tunicamycin treatment.

Chaperone HSF1 Co-Inducers Distinguished from HSF1 Stress Inducers

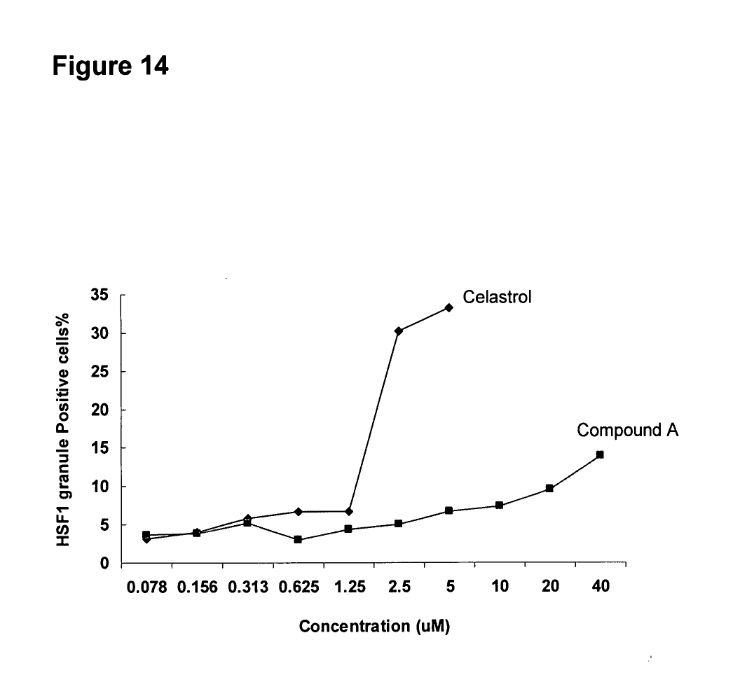

FIG. 14 shows results from an experiment comparing the percent increase of HSF1 granule positive cells in non-stressed cells (here, non-heat shocked cells cultured at 37° C. as a function of increasing concentrations of celastrol (positive control) compared to Compound A (a new MaCRA selected modulator discussed above). As shown in FIG. 14, and in contrast to celastrol (positive control), Compound A does not stimulate the heat shock response (HSF1 granules) in non-stressed cells. Compound A may thus be classified as a HSF1 co-inducer, and a co-inducer of cellular stress response, rather than being a stress inducer like celastrol. The MaCRA platform and associated methods and assays of the invention may be used to identify other members of this class of co-inducer compounds (see below).

Compound Hits from MaCRA do not Act Through Inhibition of HSP90

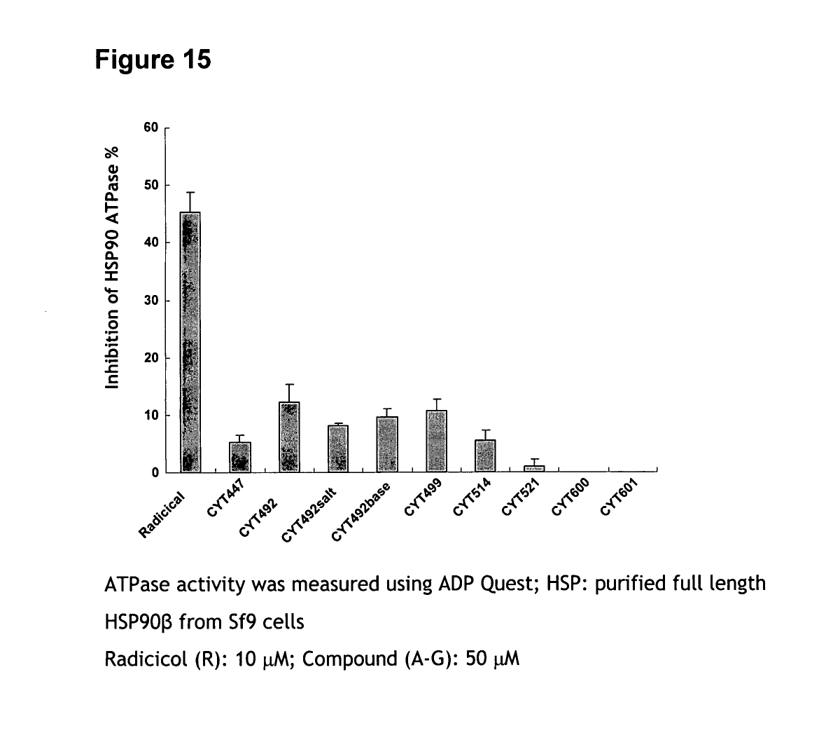

Next, it was tested whether certain selected compound hits from the MaCRA screens inhibit HSP90, which would be expected to negatively feedback on and thus inhibit HSF1 expression and thus be indirect modulators of HSF1. HSP90 (ATPase) activity was measured according to the methods in Example 8. As shown in FIG. 15, various compounds identified as hits from the MaCRA screens described above do not significantly inhibit the ATPase activity of HSP90. Thus, these compounds are modulating HSF1 through a novel mechanism that does not involve HSP90 inhibition.

### Screening Strategy for Identifying HSF1+HSP+Co-Inducers

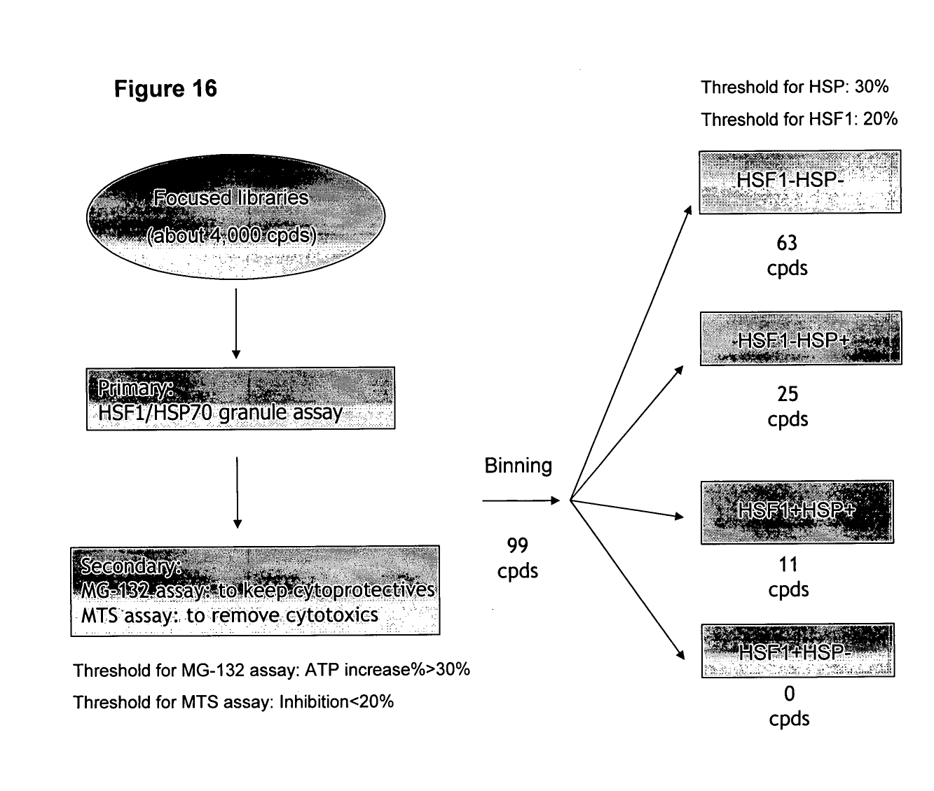

FIG. 16 is a schematic of a strategy for screening compounds for lead development using a primary HSF1/HSP70 granule assay and secondary MG-132 and MTS assays to identify cytoprotection and cytotoxicity, respectively, as described above. Based on experiments detailed above, the three above described assays were performed in parallel to screen a 4000 compound library for HSF1+HSP+co-inducers. The MG-132 assay involves treating cells with proteosome inhibitors to induce misfolded cytoplasmic proteins (which causes cellular stress and cell death). Compounds were screened for those that increase the percent of viable cells by protecting treated cells against proteosome inhibitor-induced cell death (see Example 5). The MTS assay was used to screen for (and eliminate) compounds that are generally cytotoxic to normal cells (see Example 5).

FIG. 17A represents a multidimensional compilation of data obtained from screening 4000 compounds, showing percent increase of HSF1 granule positive cells (by measuring HSF1 granule count) on the x-axis, percent increase of viable cells in the MG-132 assay on the y-axis, and percent inhibition of cell viability from the MTS assay on the z-axis. The size and the shading of the representative spheres correlate with HSP70 and HSF1 granule positive cells, respectively, as shown. Thus, darker and larger spheres are compounds that are HSF1+ and HSP70+, those further along the y-axis exhibited greater cell viability in the MG-132 assay (cytoprotection); and those further along the z-axis (higher up) exhibited greater viability in the MTS assay (cytotoxicity) than those closer to the origin. Data sorting or binning using such a multidimensional analysis enables quick use of data from parallel assays to identify modulatory compounds of interest, here, HSF1 co-inducers. These and similar multidimensional analyses may be used to display data from any number of assays. The data may be stored in a database for future use in compound screens and selections and for comparative and predictive purposes.

Multidimensional data in FIG. 17A are broken down into data from individual assays in figures as follows: FIG. 17B represents data from the HSF1 granule screen (4,000 compounds; R0=0 hour recovery time after heat shock) showing increasing percent HSF1 positive granule on the y-axis. A threshold of 20% increase for HSF1 granule positive cells was used in this screen. The shading of the squares corresponds to HSP70 granule positive cells. Accordingly, darker squares above the 20% threshold are HSF1+HSP7+ hits. FIG. 17C represents data from the HSP70 granule assay (4,000 compounds; R20=2 hour recovery time after heat shock) with a threshold of 30% for HSP70 granule positive cells. The shading of the squares corresponds to HSF1 granule positive cells. Accordingly, darker squares above the 30% threshold are HSF1+HSP7+ hits. FIG. 17D illustrates data from the MG-132 assay with a threshold of 30% for increase viable cells % (compared to DMSO) and the square shading corresponding to increased HSF1 granule positive cells.

Finally, FIG. 17E represents a compilation of MG-132 and MTS secondary assay data. As above, the size and the shading of the representative spheres correlate with HSP70 and HSF1 granule positive cells, respectively, as shown. Compounds of interest were selected as those showing at least about 30% increased cytoprotection in the MG-132 assay and more than about 20% inhibition of cytotoxicity (e.g., increased cell viability) in the MTS assay, as shown.

Tables 3-5 are summary tables with data from select compounds identified in primary and secondary assays according to the methods of the invention separated into compounds which fall in the HSF1+HSP+(A), HSF1−HSP+(B), and HSF1−HSP− (C) categories, as described above. Importantly, the assays and methods for data analyses described herein produce no compound hits that fall within the fourth possible category, i.e., HSF1−HSP70+. This confirms that screening and assay methods according to the invention (e.g., MaCRA) are HSF1-dependent and that HSF1 is the direct molecular target.

TABLE 2

| | | HSF1 | HSF1 | HSP70 | HSP | MG132 |
|---|---|---|---|---|---|---|
| | WEHI | granule | NCV | granule | TGA | % |
| Sample ID | MTS72 | @ R0 | @ R0 | @ R2 | @ R2 | increase |
| CYT1002159 | −13.31 | 35.88 | 51.33 | 87.08 | 94.70 | 77.68 |
| CYT1002239 | −79.56 | 25.74 | 23.27 | 35.70 | 58.89 | 124.02 |
| CYT1002244 | −91.77 | 40.57 | 38.68 | 58.17 | 76.90 | 166.82 |
| CYT1002282 | −7.23 | 23.04 | 13.24 | 38.31 | 61.08 | 144.72 |
| CYT1002333 | −99.65 | 36.99 | 51.08 | 81.26 | 92.57 | 68.26 |
| CYT1002357 | −16.93 | 39.14 | 36.32 | 68.55 | 81.25 | 39.98 |
| CYT1002505 | −30.13 | 19.50 | 25.65 | 37.73 | 56.52 | 44.44 |
| CYT1002532 | −46.17 | 40.26 | 47.57 | 72.82 | 85.69 | 73.88 |
| CYT1003584 | 11.21 | 17.45 | 24.34 | 42.47 | 65.86 | 46.04 |
| CYT1003666 | 3.77 | 3.56 | 27.60 | 66.27 | 84.92 | 42.00 |
| CYT1003861 | −14.31 | 25.21 | 30.63 | 33.71 | 57.24 | 60.34 |

TABLE 3

| | | HSF1 | HSF1 | HSP70 | HSP | MG132 |
|---|---|---|---|---|---|---|
| | WEHI | granule | NCV | granule | TGA | % |
| Sample ID | MTS72 | @ R0 | @ R0 | @ R2 | @ R2 | increase |
| CYT1001408 | −61.12 | 7.79 | 16.38 | 40.51 | 61.69 | 33.14 |
| CYT1001913 | 6.14 | 4.77 | 11.96 | 41.63 | 69.82 | 50.76 |
| CYT1001924 | 8.74 | 6.41 | 10.53 | 31.75 | 57.57 | 37.59 |
| CYT1001926 | 5.38 | 3.48 | 12.20 | 32.72 | 62.48 | 38.89 |
| CYT1001957 | 5.46 | 6.19 | 15.32 | 36.12 | 62.44 | 35.46 |
| CYT1001991 | −7.83 | 10.77 | 18.94 | 43.28 | 67.79 | 59.19 |
| CYT1001999 | −2.29 | 10.09 | 14.31 | 33.26 | 59.49 | 34.37 |
| CYT1002039 | −13.53 | 5.25 | 13.80 | 36.67 | 63.57 | 30.42 |
| CYT1002157 | −12.84 | 4.40 | 10.17 | 42.99 | 61.25 | 30.95 |
| CYT1002167 | −14.96 | 5.93 | 11.18 | 34.57 | 55.61 | 30.71 |
| CYT1002176 | −15.74 | 5.59 | 13.32 | 33.56 | 54.22 | 58.56 |
| CYT1002185 | −12.91 | 7.65 | 14.23 | 33.96 | 53.37 | 30.12 |
| CYT1002193 | −9.41 | 7.61 | 12.22 | 36.09 | 56.11 | 30.95 |
| CYT1002207 | −11.12 | 14.71 | 14.67 | 44.89 | 66.65 | 44.10 |
| CYT1002288 | −71.31 | 16.04 | 13.44 | 37.92 | 59.66 | 68.65 |
| CYT1002314 | −90.19 | 13.77 | 9.36 | 43.07 | 63.59 | 53.03 |
| CYT1002365 | −29.02 | 7.31 | 12.72 | 41.31 | 61.68 | 32.50 |
| CYT1003175 | −63.52 | 7.87 | 13.93 | 31.78 | 54.46 | 34.54 |
| CYT1003276 | −49.24 | 8.69 | 7.93 | 30.60 | 54.08 | 36.25 |
| CYT1003434 | −51.90 | 12.05 | 12.54 | 31.26 | 51.55 | 47.81 |
| CYT1003747 | 4.22 | 7.04 | 9.68 | 34.34 | 61.40 | 32.31 |
| CYT1003909 | 13.56 | 9.44 | 12.62 | 54.58 | 75.27 | 38.50 |
| CYT1003921 | 0.68 | 5.93 | 7.15 | 34.10 | 58.77 | 33.50 |
| CYT1003976 | −4.53 | 6.21 | 10.47 | 39.26 | 65.29 | 40.85 |
| CYT1004053 | 4.24 | 5.67 | 9.82 | 32.01 | 56.98 | 36.11 |

TABLE 4

| | | HSF1 | HSF1 | HSP70 | HSP | MG132 |
|---|---|---|---|---|---|---|
| | WEHI | granule | NCV | granule | TGA | % |
| Sample ID | MTS72 | @ R0 | @ R0 | @ R2 | @ R2 | increase |
| CYT1000996 | 1.74 | 9.75 | 18.51 | 16.91 | 33.76 | 31.69 |
| CYT1001439 | −157.96 | 4.86 | 5.43 | 29.89 | 54.63 | 31.26 |
| CYT1001541 | −21.12 | 1.87 | 3.45 | 16.22 | 34.11 | 41.28 |
| CYT1001558 | 0.24 | 3.57 | 5.55 | 14.40 | 26.82 | 73.20 |
| CYT1001821 | −139.62 | 7.98 | 7.25 | 21.53 | 42.80 | 39.05 |
| CYT1001838 | −26.26 | 1.69 | 3.59 | 20.42 | 41.54 | 39.67 |
| CYT1001876 | −46.16 | 2.48 | 2.24 | 9.92 | 25.08 | 40.93 |
| CYT1001929 | 6.69 | 4.72 | 10.60 | 28.51 | 54.55 | 44.58 |
| CYT1002241 | −37.26 | 9.55 | 5.25 | 23.20 | 47.88 | 40.44 |
| CYT1002245 | −79.91 | 10.02 | 7.69 | 27.72 | 48.05 | 44.16 |
| CYT1002247 | −65.37 | 4.24 | 6.57 | 18.73 | 38.82 | 30.26 |
| CYT1002255 | −87.42 | 9.31 | 8.20 | 21.89 | 40.38 | 36.39 |
| CYT1002258 | −81.60 | 5.44 | 6.69 | 19.03 | 34.18 | 45.81 |
| CYT1002261 | −87.01 | 11.86 | 8.72 | 24.42 | 44.82 | 59.26 |
| CYT1002262 | −81.53 | 8.50 | 6.32 | 26.54 | 46.81 | 52.93 |
| CYT1002264 | −79.09 | 10.86 | 15.08 | 26.81 | 45.33 | 36.56 |
| CYT1002265 | −73.95 | 15.45 | 12.95 | 29.55 | 50.02 | 59.32 |
| CYT1002266 | −38.46 | 17.56 | 12.42 | 28.85 | 48.30 | 43.63 |
| CYT1002268 | −94.30 | 6.59 | 10.33 | 22.04 | 41.86 | 31.61 |
| CYT1002289 | −89.54 | 10.03 | 10.82 | 27.68 | 47.54 | 52.84 |
| CYT1002404 | −24.18 | 5.94 | 12.65 | 26.19 | 50.88 | 31.53 |
| CYT1002408 | −22.07 | 3.50 | 11.09 | 24.14 | 48.70 | 34.72 |
| CYT1002477 | −46.00 | 4.30 | 8.64 | 20.63 | 44.09 | 37.50 |
| CYT1002478 | −43.79 | 3.40 | 7.60 | 22.27 | 45.49 | 35.10 |
| CYT1002515 | −43.43 | 5.33 | 7.50 | 19.76 | 42.52 | 33.26 |
| CYT1002520 | −44.76 | 7.11 | 10.21 | 22.32 | 46.09 | 32.96 |
| CYT1002652 | −6.16 | 5.87 | 9.45 | 26.02 | 50.97 | 48.55 |
| CYT1002671 | −4.76 | 5.76 | 6.31 | 27.16 | 49.03 | 53.62 |
| CYT1002682 | −4.82 | 3.94 | 4.74 | 15.93 | 37.95 | 34.90 |
| CYT1002785 | −34.67 | 7.38 | 8.26 | 23.17 | 45.92 | 32.32 |
| CYT1002804 | −27.28 | 8.26 | 6.75 | 16.36 | 40.46 | 33.29 |
| CYT1002817 | −47.30 | 6.18 | 5.55 | 11.53 | 30.66 | 48.06 |
| CYT1002840 | −33.32 | 7.03 | 7.78 | 12.05 | 33.76 | 36.38 |
| CYT1002871 | −3.99 | 10.98 | 16.89 | 21.54 | 45.63 | 58.49 |
| CYT1002896 | −36.91 | 6.55 | 11.38 | 23.71 | 46.03 | 40.00 |
| CYT1002904 | −41.12 | 6.77 | 11.11 | 24.59 | 49.89 | 50.57 |
| CYT1002930 | −23.21 | 7.10 | 9.89 | 21.15 | 43.62 | 34.67 |
| CYT1002953 | −48.99 | 6.38 | 9.58 | 24.91 | 49.80 | 35.33 |
| CYT1002955 | −49.54 | 5.29 | 7.90 | 27.46 | 55.66 | 43.98 |
| CYT1002974 | −53.33 | 5.36 | 8.29 | 25.03 | 49.92 | 35.99 |
| CYT1003060 | −25.28 | 13.25 | 17.16 | 28.31 | 52.63 | 56.62 |
| CYT1003141 | 0.67 | 19.45 | 13.55 | 29.91 | 51.48 | 46.71 |
| CYT1003246 | −38.18 | 13.09 | 10.28 | 27.71 | 52.77 | 31.63 |
| CYT1003288 | −15.46 | 5.25 | 6.56 | 26.43 | 49.93 | 33.17 |
| CYT1003319 | −39.49 | 4.44 | 5.77 | 21.90 | 44.23 | 59.46 |
| CYT1003389 | −6.57 | 7.47 | 8.79 | 22.97 | 46.44 | 32.55 |
| CYT1003409 | 12.02 | 9.78 | 12.51 | 29.40 | 52.83 | 37.91 |
| CYT1003437 | −0.33 | 7.68 | 8.79 | 16.63 | 35.71 | 30.22 |
| CYT1003438 | 1.66 | 7.57 | 9.07 | 18.56 | 37.46 | 33.11 |
| CYT1003455 | −25.27 | 6.53 | 7.76 | 17.08 | 35.63 | 30.14 |
| CYT1003469 | −3.06 | 6.66 | 8.20 | 15.80 | 36.21 | 36.78 |
| CYT1003478 | −0.37 | 7.31 | 8.19 | 18.95 | 40.23 | 31.64 |
| CYT1003487 | 0.19 | 10.12 | 8.70 | 18.28 | 38.25 | 34.50 |
| CYT1003537 | −5.29 | 9.54 | 14.36 | 24.47 | 49.74 | 38.96 |
| CYT1003538 | −66.13 | 8.68 | 12.86 | 24.10 | 48.41 | 37.01 |
| CYT1003691 | 12.62 | 6.80 | 10.74 | 22.01 | 44.57 | 38.41 |
| CYT1003717 | 0.21 | 5.25 | 6.48 | 20.35 | 46.42 | 38.52 |
| CYT1003938 | 9.78 | 5.78 | 9.39 | 25.36 | 50.54 | 40.07 |
| CYT1003944 | 0.96 | 3.78 | 6.23 | 25.83 | 52.12 | 46.54 |
| CYT1003946 | 4.08 | 5.97 | 5.22 | 27.65 | 53.26 | 54.15 |
| CYT1003948 | 12.10 | 7.40 | 10.47 | 25.43 | 52.12 | 31.65 |
| CYT1003997 | 2.36 | 4.60 | 11.31 | 23.25 | 45.49 | 44.38 |
| CYT1004071 | 1.00 | 12.15 | 12.91 | 27.94 | 48.58 | 32.61 |

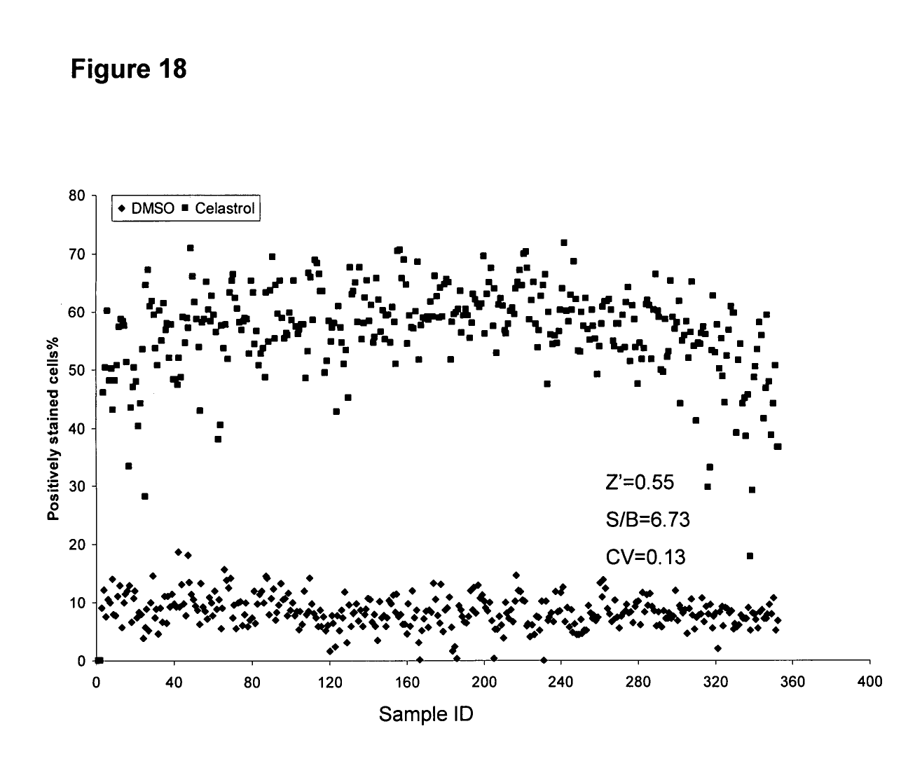

The above described experiments were performed in a 96-well format to optimize assay conditions and validate data sorting. Next, compounds were screened at a higher throughput (384-well format), again using celastrol as a positive control to see how the MaCRA platform performs at a higher throughput in a 384-well format (Example 10). FIG. 18 shows a 384-well plate evaluation of HeLa cells pre-treated with DMSO (♦) and celastrol (▪) and subsequently heat shocked at 41° C. for 2 hours with no recovery time using HSF1 granule count. The data show that all screening criteria from the 96-well format are met when MaCRA is scaled up to the 384-well format (Z′=0.55, signal-to background ratio (S/B)=6.73 and CV=0.13).

### MaCRA Screen HSF1+ Compound Hits are HSF1-Dependent

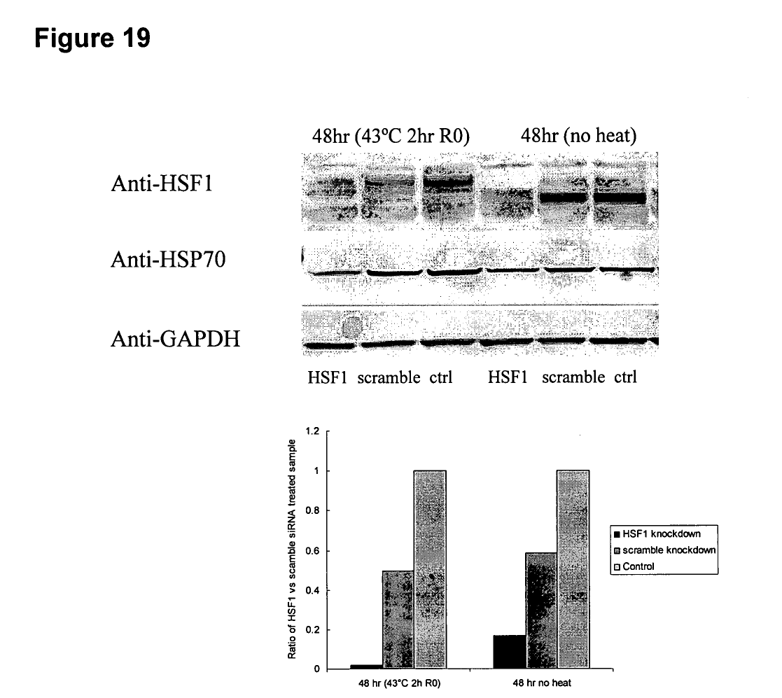

To verify that the above described MaCRA identified HSF1 activating compounds indeed act directly through HSF1, a series of RNA interference (RNAi) knock down experiments were performed to look at the effect of those compounds when HSF1 expression levels are directly reduced in cells by administering siRNA constructs specific for HSF1 compared to non-specific control siRNAs (see Example 11). FIG. 19 shows a Western blot for HSF1 and HSP70 protein expression (GAPDH protein expression as a loading control) in siRNA treated HeLa cells transfected with 25 nM of HSF1 siRNA, scramble siRNA or a transfection control, for 48 h followed by 43° C. heat shock for 2 hours (2 h) with no recovery time (R0) or a non-heat shock treatment. FIG. 19 depicts a bar chart including the ratio of expression of HSF1, scramble, and GAPDH in HSF1 siRNA treated compared to control (scramble) siRNA treated samples. Under these conditions, HSF1 expression was reduced to about 80-90% and HSP70 expression was reduced to about 50% of control levels.

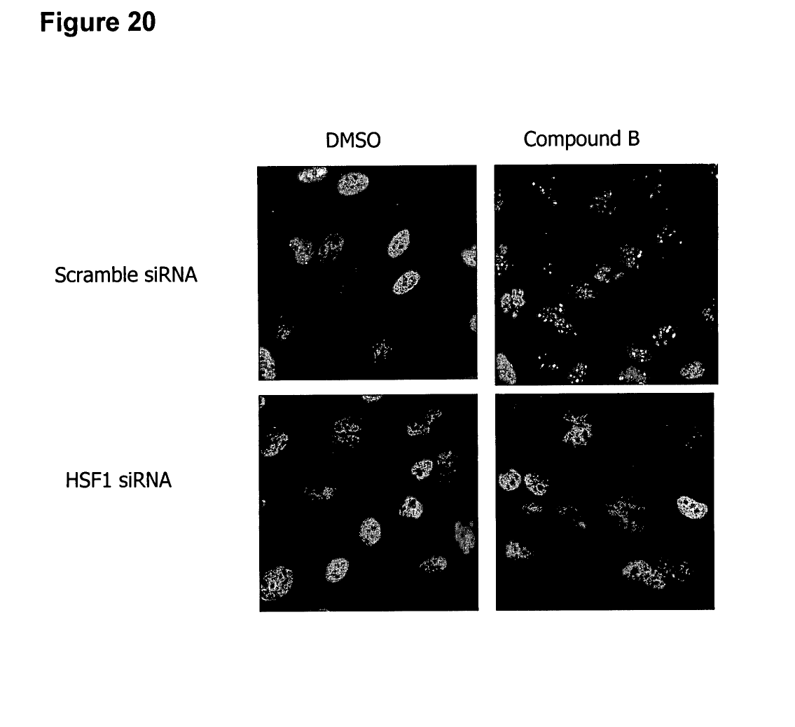

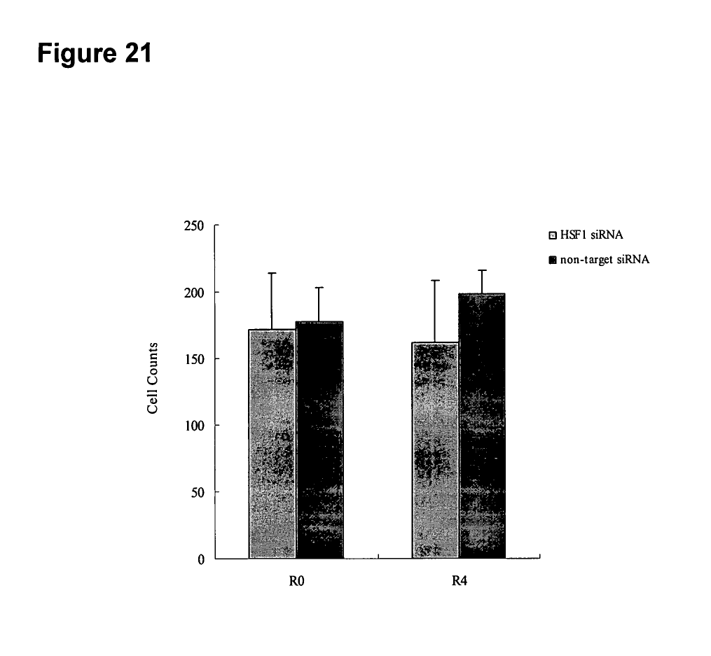

FIG. 20 looks at the effect of HSF1 knock down on HSF1 positive granule formation in HeLa cells treated with 41° C. heat shock for 2 h or non-heat shock treatment transfected with 25 nM HSF1 siRNA and scramble siRNA for 48 h followed by treatment with 25 μM Compound B (CYT492) or a DMSO control before the treatment. Granule formation is seen in control (scramble) siRNA treated cells but not in HSF1 siRNA-specific siRNA treated cells. This shows that HSF1 positive granule formation is directly dependent on HSF1 expression in a cell. To rule out that the above siRNA treatments are not simply killing treated cells, cell counts of HeLa cells transfected with 25 nM HSF1 siRNA and scramble (non-target) siRNA were measured (FIG. 21). This experiment confirms that HSF1 siRNA transfection in HeLa cells does not cause nonspecific cell death.

Table 5 shows compiled data from such siRNA knock down experiments using nine independent hits from the HSF1+HSP70+ category that were also identified as co-inducers (amplifiers) of HSF1. As shown in Table 5, each of these compounds is an HSF1-dependent activator.

TABLE 5
HSF1-Dependent Activators (siRNA experiments)

| | | EC50 | EC50 | |
|---|---|---|---|---|
| | EC50 | (41° C. 2 h, | (41° C. 2 h, | EC50 |
| | (HSF1) | scramble | HSF1 | (No heat |
| ID | (41° C. 2 h) | siRNA) | siRNA) | shock) |
| CYT1 | 14.27 | 15.66 | No activity up | No activity up |
| | | | to 80 μM | to 80 μM |
| CYT2 | 31.20 | 41.89 | No activity up | No activity up |
| | | | to 80 μM | to 80 μM |
| CYT3 | 11.81 | 17.44 | No activity up | No activity up |
| | | | to 80 μM | to 80 μM |
| CYT4 | 10.85 | 24.72 | No activity up | No activity up |
| | | | to 80 μM | to 80 μM |
| CYT5 | 10.44 | 13.1 | No activity up | No activity up |
| | | | to 80 μM | to 80 μM |
| CYT6 | 14.39 | 14.53 | No activity up | No activity up |
| | | | to 80 μM | to 80 μM |
| CYT7 | 22.6 | 24.83 | No activity up | No activity up |
| | | | to 80 μM | to 80 μM |
| CYT8 | 10.28 | 10.49 | No activity up | No activity up |
| | | | to 80 μM | to 80 μM |
| CYT9 | 19.21 | 50.9 | No activity up | No activity up |
| | | | to 80 μM | to 80 μM |

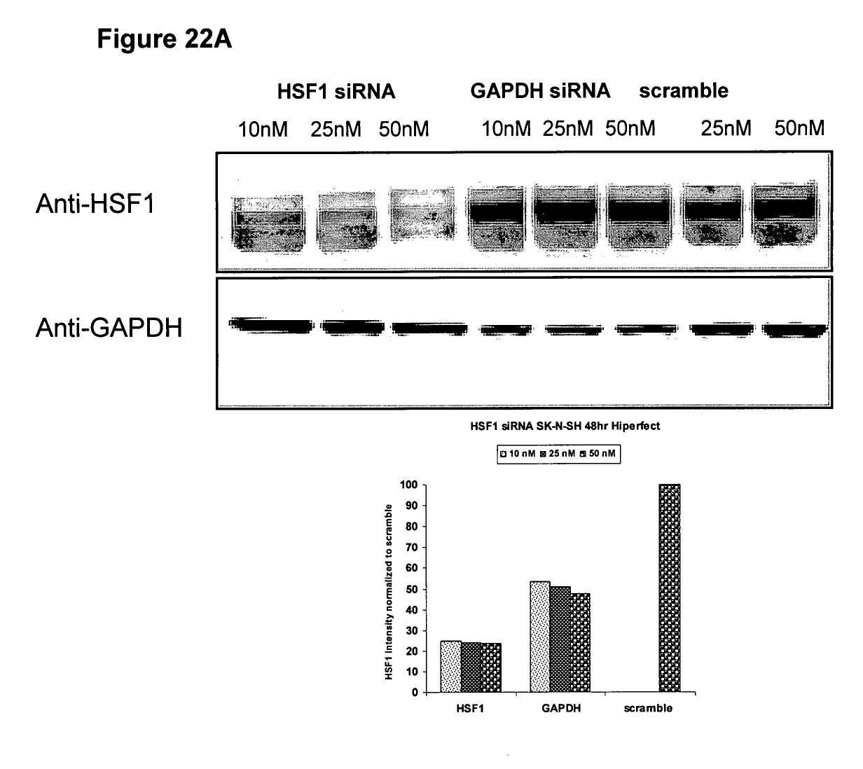

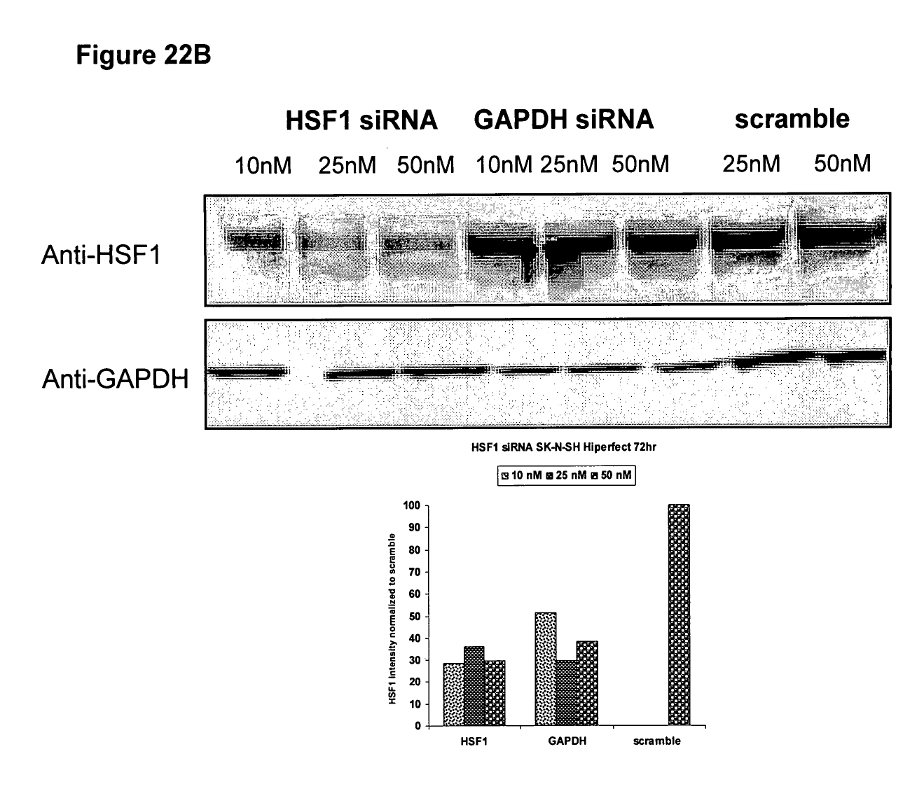

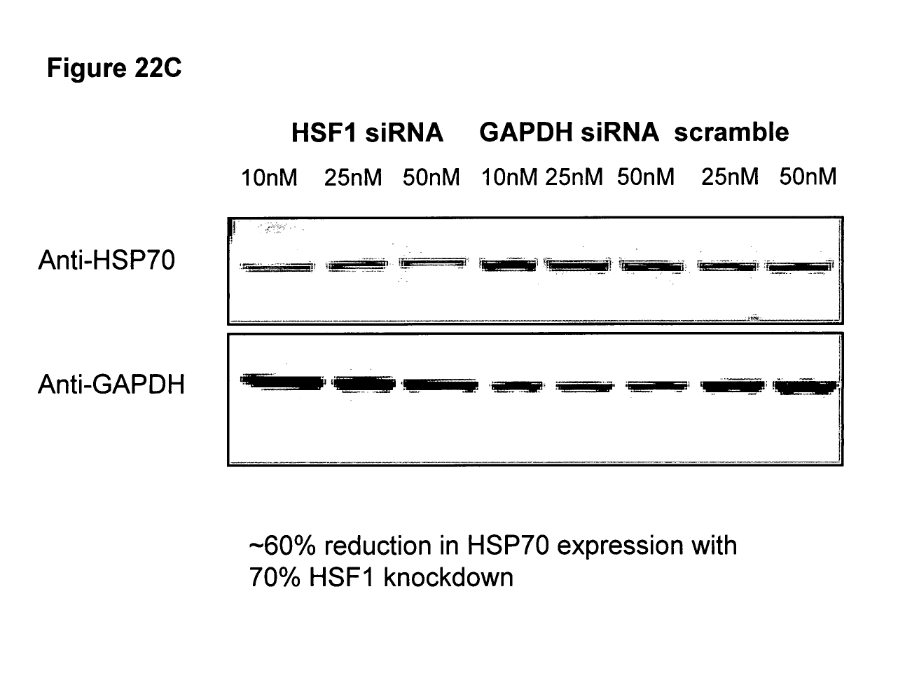

Next, MaCRA-selected compounds were tested in siRNA knock down experiments followed by functional secondary assays (MTS and MG-132 assays, see Example 5). FIGS. 22A-B illustrate an siRNA knockdown of HSF1 in SK-N-SH cells used in the MG-132 assay with 10, 25 and 50 nM of HSF siRNA against GAPDH siRNA (control) and scramble siRNA (control) for 48 h (A) and 72 h (B) and a corresponding Western blot. FIG. 22C shows a Western blot illustrating the effects on HSP70 expression after HSF1 knockdown for 48 hours with 10, 25 and 50 nM of HSF1 siRNA compared with GAPDH siRNA and scramble siRNA. Here, it was observed that HSF1 knock down was not as efficient as that seen in HeLa cells (above). HSF1 siRNA knockdown in SK-N-SH cells resulting in about 70% knock down of HSF1 and about 60% knock down of HSP70 in SK-N-SH cells. Nonetheless, the knock down levels achieved in SK-N-SH cells permitted us to test whether the compound hits act directly through HSF1 in the MG-132 assay.

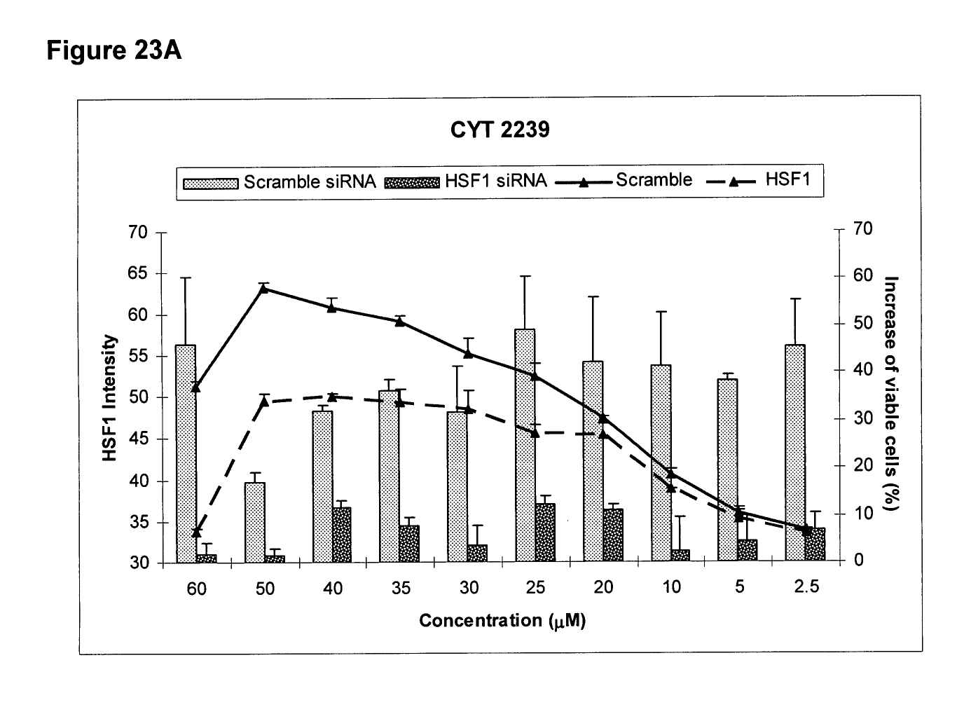

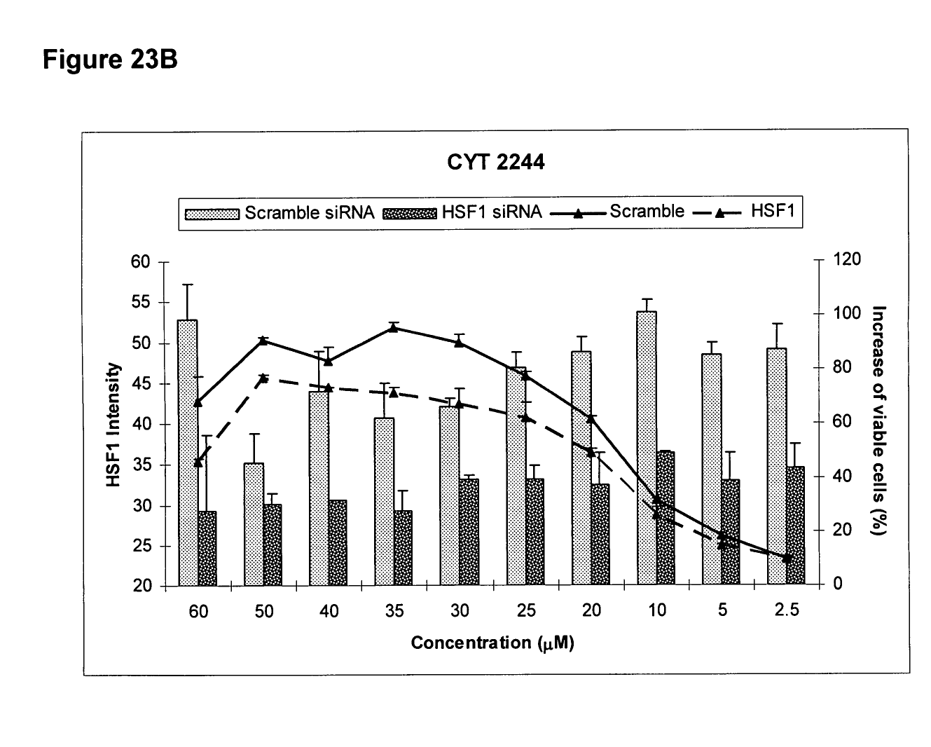

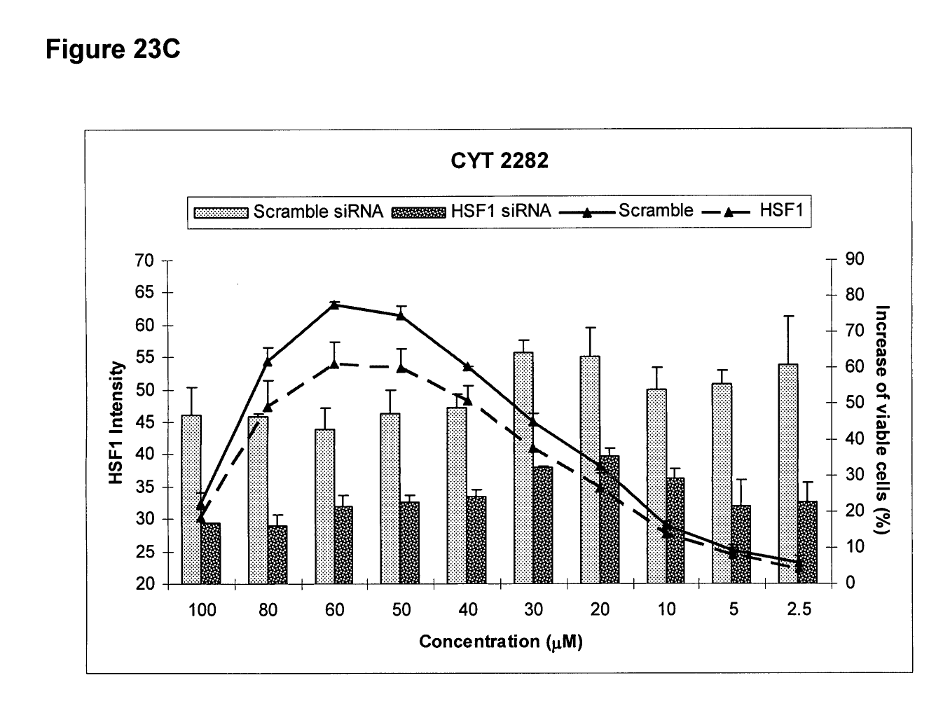

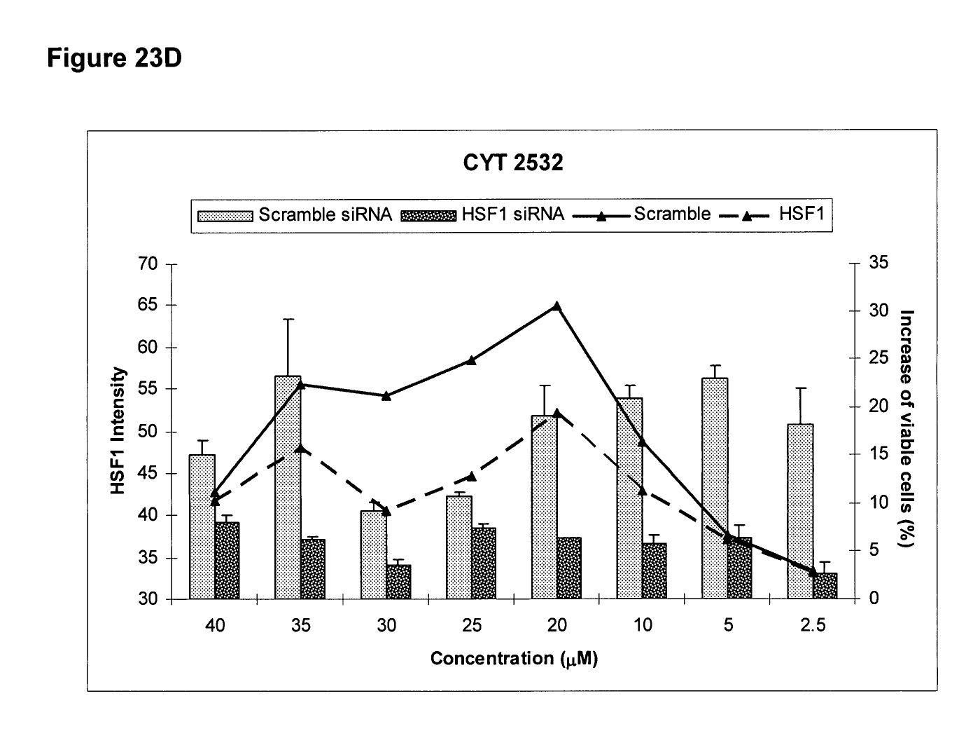

FIGS. 23A-D show HSF1 dependent cytoprotection of SK-N-SH cells in the MG-132 assay when treated with 50 nM HSF1 siRNA and scramble siRNA for 48 h following pretreatment with one of compounds CYT 2239 (A), CYT 2244 (B), CYT2282 (C) or CYT 2532 (D). The four test compounds showed HSF1 dependent cytoprotection. When HSF1 levels were reduced by siRNA knock down, there was about a 10-20% reduction of cytoprotection in the MG-132 assay.

### Use of HSF1 Granule Assays to Identify Inhibitors of Cell Stress Response

Identification of HSF1 inhibitors would be desirable as they could be used in treatments relating to inhibition of cell growth such as in methods for targeted or regulated cell death and in cancer therapeutics, for example. Triptolide is a known HSF1 inhibitor for cancer therapeutics. See, e.g., Phillips et al. Cancer Research (2007) 67, 9407; Westerheide et al., J. Biol. Chem. (2006) 281, 9616; Dai et al., Cell 2007; 130(6):1005-18. To date, however, there have been no reported methods, and certainly no quantifiable and high throughput methods, to screen and select putative HSF1 inhibitory compounds. Triptolide was used as a positive control in the HSF1 granule assays and secondary assays described above to tailor the MaCRA format for selection of HSF1 inhibitory compounds (modulators that reduce HSF1 activity in the cell). Accordingly, inhibition of HSF1 granule formation by increasing amounts of triptolide was tested at four different treatment times (1-4 hours) at 43° C. with no recovery time (Example 12). It was observed that 43° C. with no recovery time offers greater sensitivity for selection of HSF1 inhibitors (as compared to activator selections in which greater sensitivity was seen with 41° C. heat shocks).

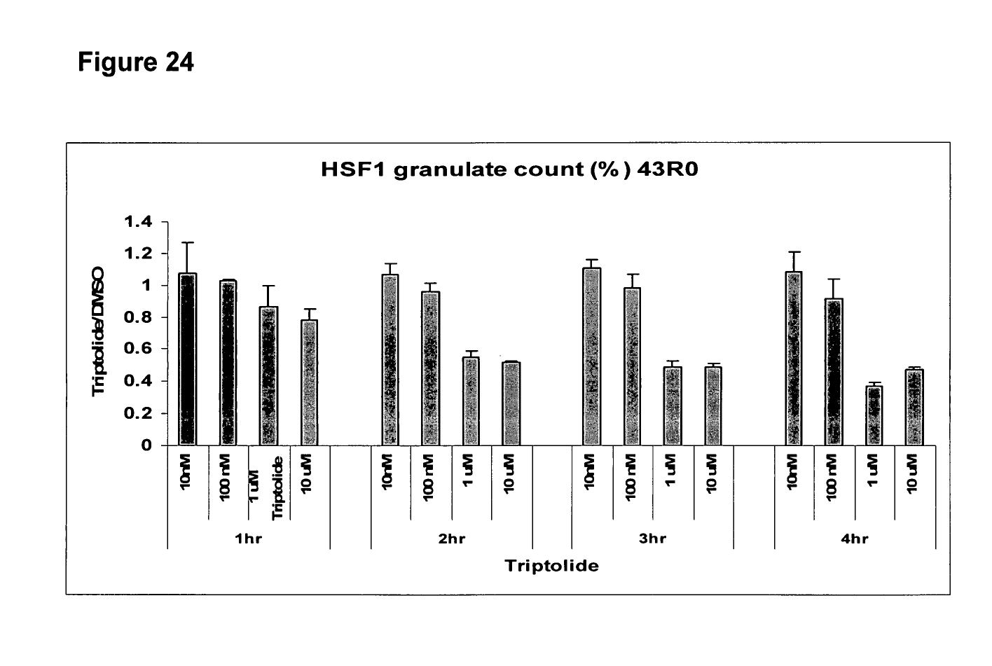

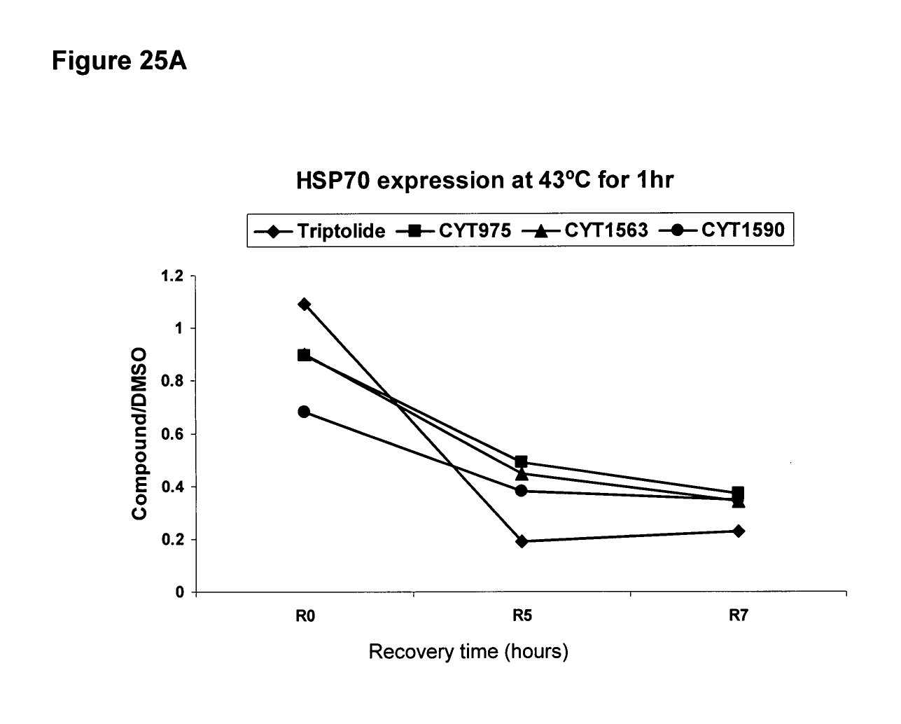

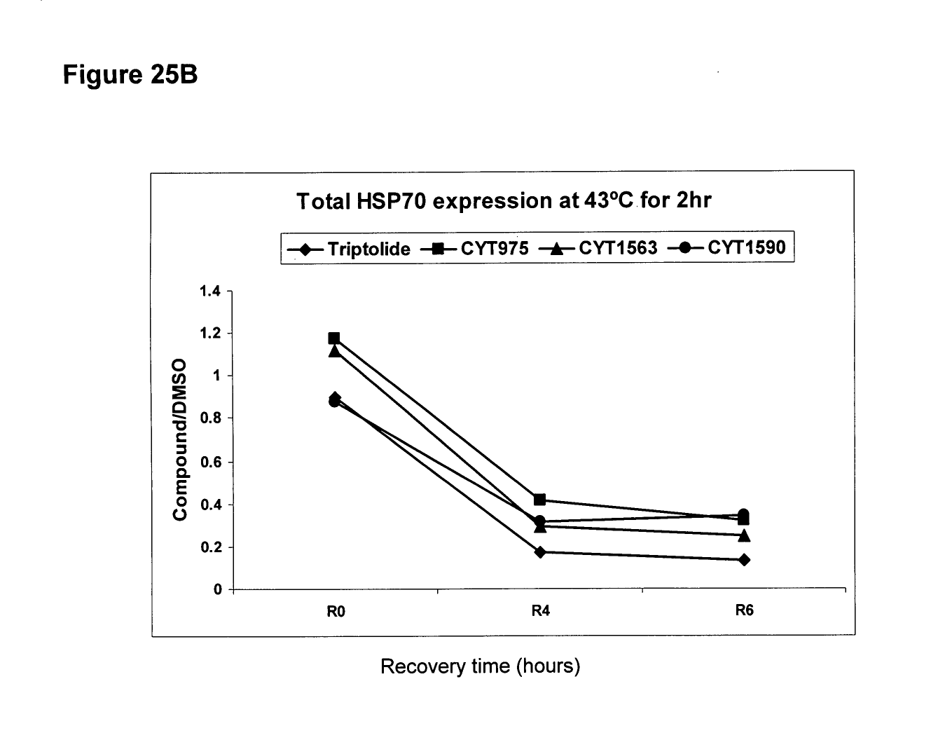

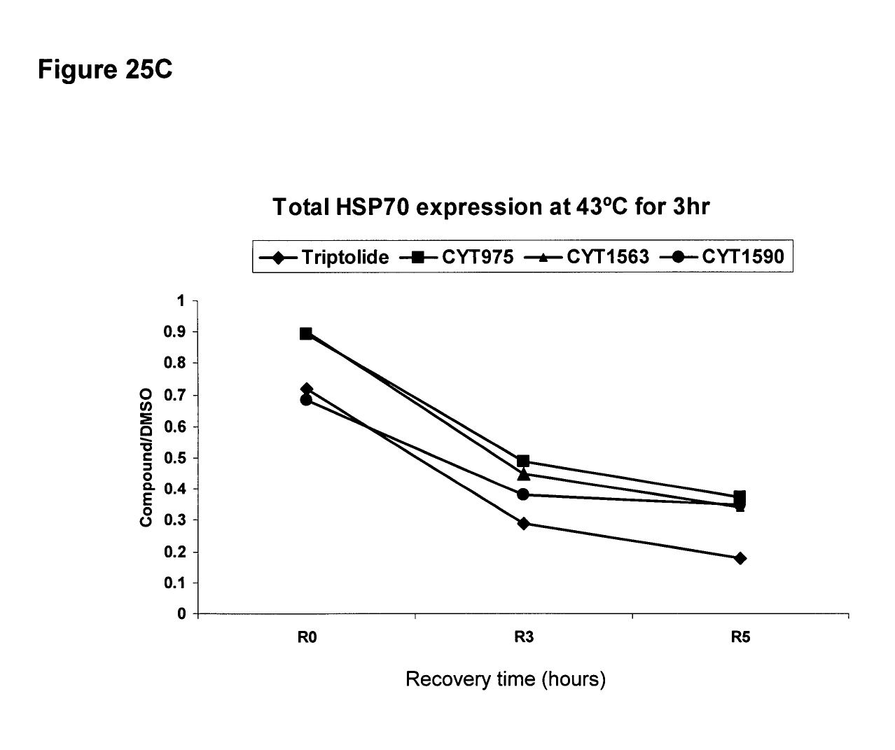

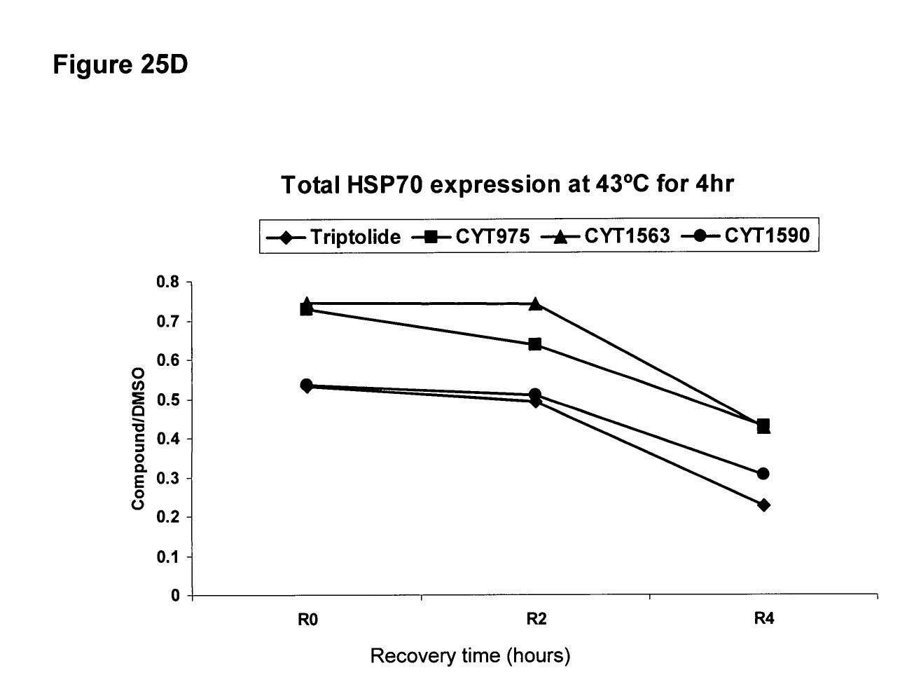

FIG. 24 illustrates the dose dependent inhibition of HSF1 granule formation in HeLa cells treated with increasing concentrations of triptolide (10 nM, 100 nM, 1 μM and 10 μM) after a 43° C. heat shock for 1, 2, 3 or 4 hours. HSF1 granule count was measured with 5 granules/nucleus used as the threshold. Similar assay conditions were used to test the effect of various compounds selected using MaCRA methods as described herein. FIGS. 25A-D show the reduction of HSP70 expression in HeLa cells treated with 1 μM triptolide (♦), 10 μM CYT 975 (▪), 10 μM CYT 1563 (▴) or 10 μM CYT 1590 () at 43° C. heat shock for 1, 2, 3 or 4 hours with 0, 5 or 7 hours recovery time with total HSP70 nuclear and cell intensity as the threshold.

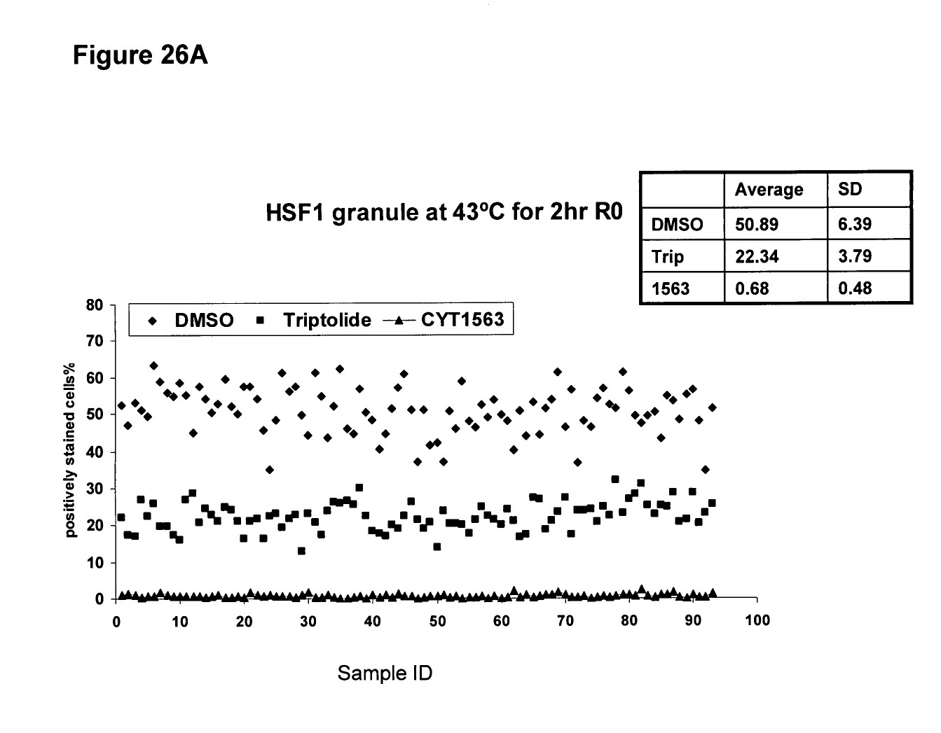

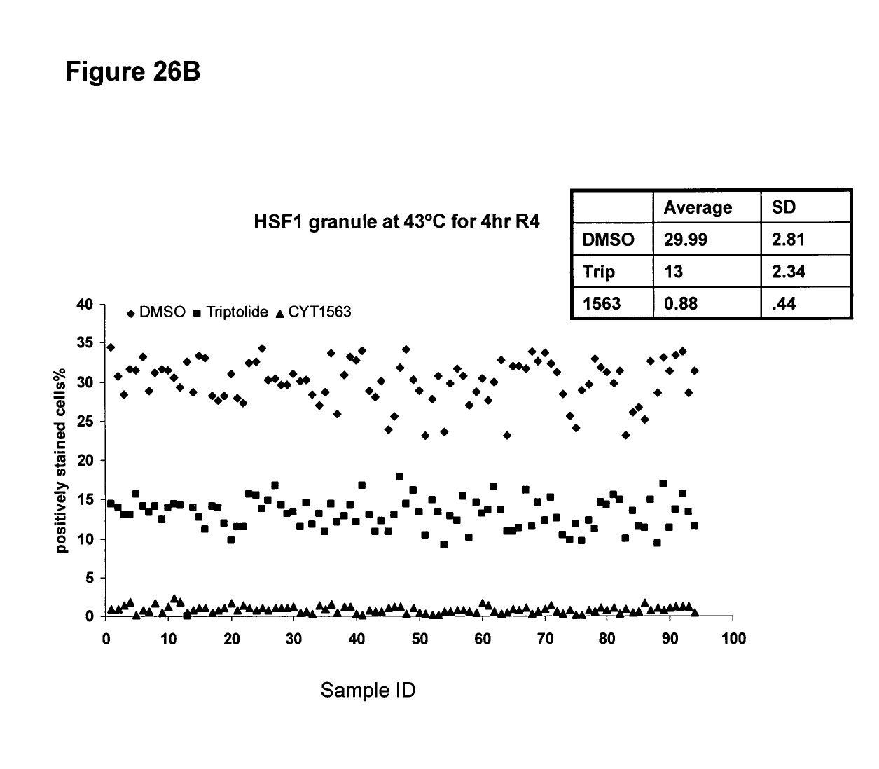

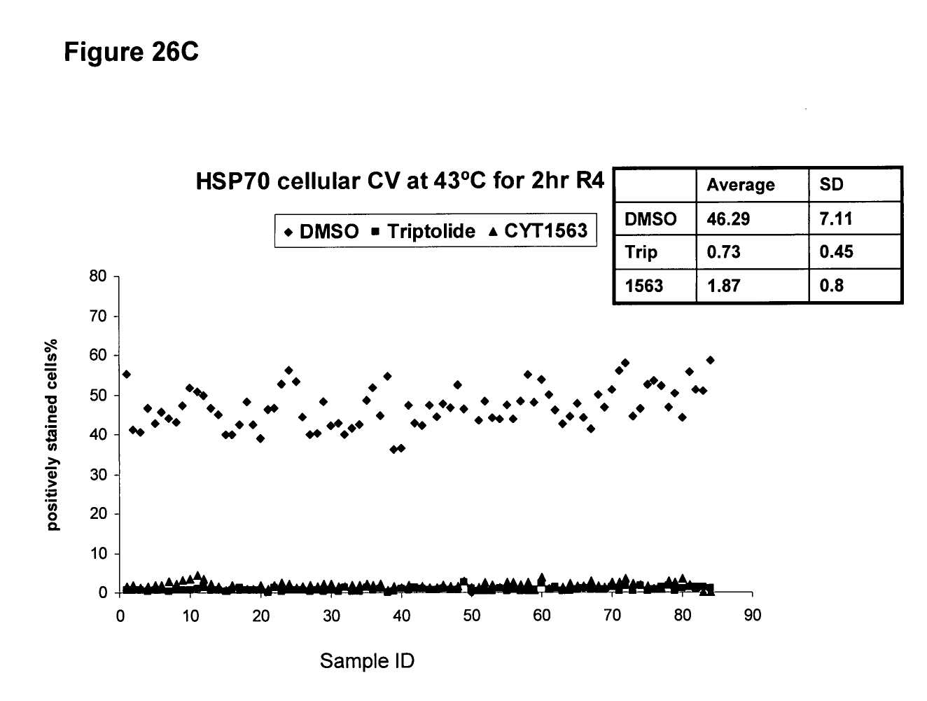

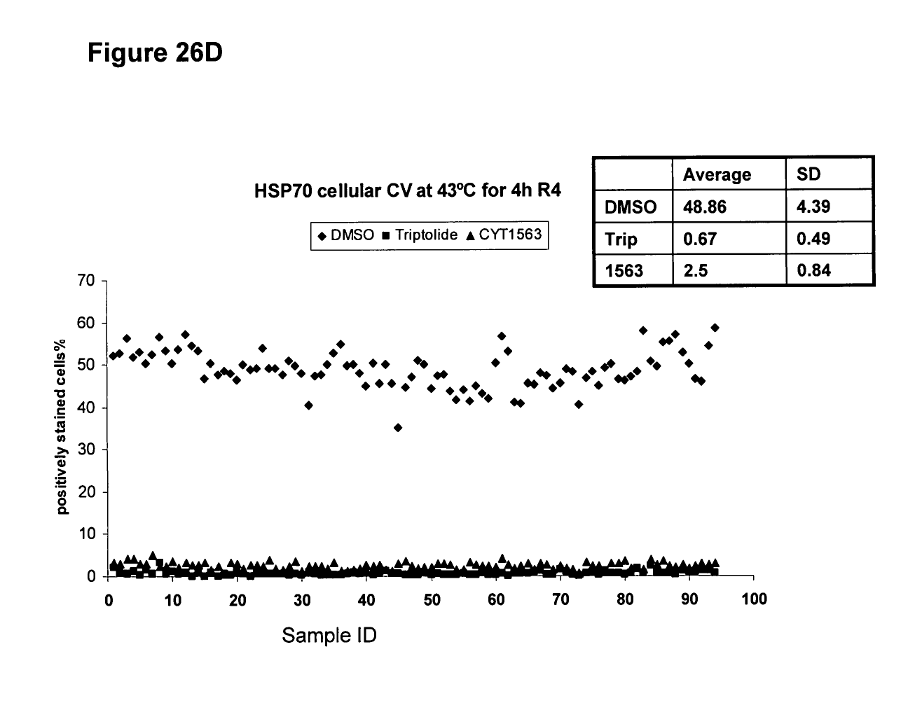

Based on the data above, the following four conditions for inhibition of HSF1 and HSP70 were selected for follow-up study in the MaCRA platform (Example 13): 1) 43° C. for 2 hours at R0 for HSF1 inhibition (FIG. 26A); 2) 43° C. for 4 hours at R4 for HSF1 inhibition (FIG. 26B); 3) 43° C. for 2 hours at R4 for HSP70 inhibition (FIG. 26C); and 4) 43° C. for 4 hours at R4 for HSP70 inhibition (FIG. 26D). As positive controls, 1 μM triptolide and 10 μM of CYT1563 were used.

Data from these studies are summarized in Table 6 below with Z′ calculated for triptolide and CYT1563. Because Z′ for CYT1563 are good (Z′>0.5 being considered as robotic) in HSF1 and HSP70 granule assays at 43° C. for 4 hours at R4, this condition was selected for further HSF1/HSP70 inhibitor screening.

TABLE 6

| | Z′ | Z′ |
|---|---|---|
| Heat shock type | Triptolide | CYT1563 |
| 43° C. 2 hour R0 | <0 | 0.28 |
| HSF1 granule | | |
| positive cells % | | |
| 43° C. 2 hour R4 | 0.50 | 0.47 |
| HSP70 granule | | |
| positive cells % | | |
| 43° C. 4 hour R4 | 0.09 | 0.70* |
| HSF1 granule | | |
| positive cells % | | |
| 43° C. 4 hour R4 | 0.09 | 0.66* |
| HSP70 granule | | |
| positive cells % | | |

*43° C. for 4 hours, with a 4 hour recovery time (R4) was selected as the optimal condition for further HSF1 and HSP70 inhibitor screening.

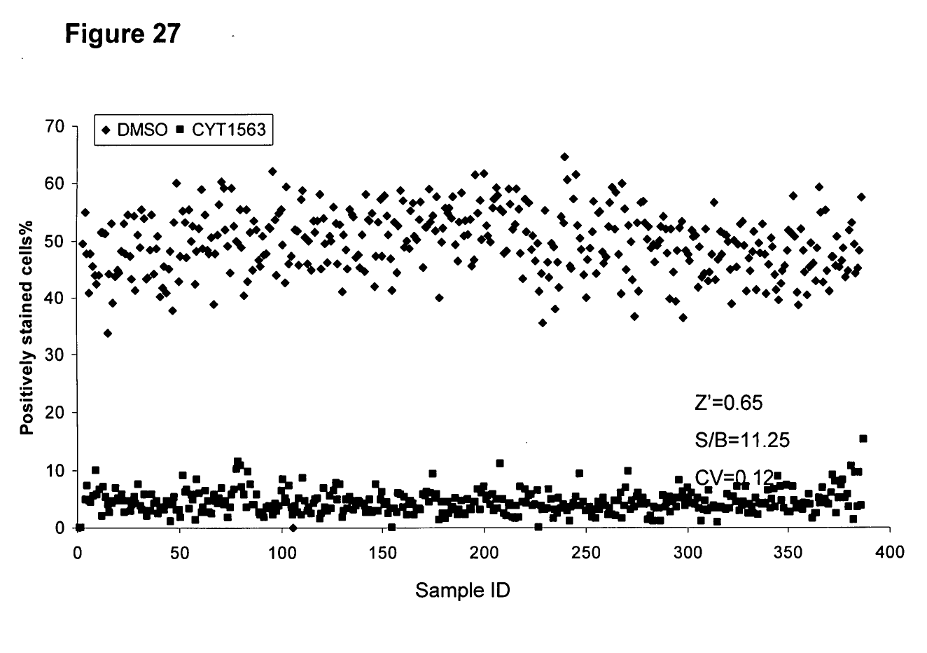

In the next step, the MaCRA based HSF1/HSP inhibitor screening assay was scaled up from a 96-well to a 384-well format using the above optimized conditions (Example 14). In this experiment, a unique data binning strategy similar to the one used above to identify HSF1 co-inducers was employed, except that here, compounds having no inhibitory activity in the absence of cellular stress but showing HSF1 specific inhibitory activity upon cellular stress (i.e., heat shock) were sought. FIG. 27 shows a 384-well plate evaluation of HeLa cells treated with DMSO only (♦) or CYT 1563 (10 μM) (▪) and subsequently heat shocked at 43° C. for 2 h with no recovery time using HSF1 granule count. A Z′ of CYT1563 at 0.65, Signal/Background (S/B) ratio of 11.25 and CV of 12% were observed.

One of skill in the art will recognize that the above described assays and data binning strategies may be varied to fit the particular situation. In general, the parameters are varied individually and together to optimize the screen based on particular cells, compounds and assay conditions. The foregoing examples are presented for illustrative purposes only, and are not intended to be limiting. One of skill in the art will recognize that additional embodiments according to the invention are contemplated as being within the scope of the foregoing generic disclosure, and no disclaimer is in any way intended by the following, non-limiting examples.

### EXAMPLES

### Example 1

### Quantification of HSF1/HSP Stress Granules and Assay Validation

This experiment was directed to developing of a HSF1/HSP70 high-content screening (HCS) assay for screening HSF1 activators. HeLa cells were pretreated with a compound (celastrol) one hour before heat shock at 41° C. for two hours. The dilution in Dulbecco's Modified Eagle's Medium (DMEM) was 200-fold resulting in a final concentration of 30 μM for screening. A custom made aluminum plate was designed for better heat transduction and to achieve a constant temperature with minimal variability for the 96-well plate. The aluminum plate was placed inside a 41° C. incubator for one day prior to the experiment.

Immunocytochemical staining for HSF1 and HSP70 in HeLa cells was performed as in Zhang et al., Biomol Screen., 13(6):538-543, 2008. Image acquisition was performed using an INcell 1000 (GE Healthcare, Piscataway, N.J.) integrated with a Twister II (Caliper Life Sciences, Hopkinton, Mass.) for automated plate delivery. Image analysis was carried out using a Multi Target Analysis module from Workstation 3.6. Algorithms for HSF1/HSP70 granule count, granule area and nuclear intensity CV were established according to assay conditions and manufacture instructions. EC₅₀ values and curve fitting were performed using a Prism 4.0 (GraphPad Software, San Diego, Calif.) with nonlinear regression analysis. Celastrol (2 μM) induced granule formation was observed in treated (FIGS. 1A and 1C) but not in DMSO solvent-treated control cells (FIGS. 1B and 1D).

A Multi Target Analysis Module (MTA) module from Workstation software (GE Healthcare) provides a high-speed measurement of nuclear granules including granule count, granule area, granule intensity and nuclear intensity CV (CV of pixel intensity in the nucleus). FIGS. 2A and 2B provide a quantification of HSF1 variables via granule count and nuclear intensity CV. FIGS. 2C and 2D adopted granule count and granule area for the quantification of HSP70 granules.

FIG. 2A shows that heat shock (at 41° C. for two hours) induced, on average, 5.34±0.72 HSF1 stress granules per nucleus in HeLa cells exposed to 2 μM of celastrol as quantified by MTA. By comparison, DMSO-treated cells contain, on average, 2.46±0.22 granules. To achieve a relatively low background, HeLa cells that contained more than 5 HSF1 granules were designated as “HSF1 granule positive cells”. The thresholds for HSF1 nuclear intensity CV, HSP70 granule count and HSP70 granule area were also chosen with gating values equivalent to the average of DMSO treated samples plus 2 or more standard deviations (FIG. 2B-2D).

The HCS granule assay was further validated for robotic high throughput operation with Sciclone liquid handling system (Caliper Life Sciences, Hopkinton, Mass.). Celastrol treated samples from 20 HCS assay plates were collected for calculating Z′ with an average value of 0.62, demonstrating a reasonably performed robotic assay. Evaluation of HCS granule assay performance on Sciclone ALH3000 provided primary screening data as shown in FIG. 3 using HSF1 granule count. An average of 59.36%±4.71% HSF1 granule positive cells was observed with a tight CV value (7.94%) compared to that of DMSO controls at 8.17%±2.00%. The signal-to-noise ratio of celastrol is 7.13, indicating a significantly improved assay window when heat shock experiments were conducted at 41° C. (comparing to 43° C., data not shown).

FIGS. 4A and 4B provide the EC₅₀ values for celastrol and Compound A induced HSF1 and HSP70. The threshold used in determining the HSF1 EC₅₀ was nuclear intensity CV while the threshold used in determining the HSP70 EC₅₀ was total granule area.

To compare the kinetics of compound A and celastrol on HSF1/HSP70 induction, we performed a detailed time course study (up to 6 hours in the recovery time) as illustrated in FIG. 5. The concentrations used in this study were 1 μM and 10 μM for celastrol and Compound A, respectively, which are close to their EC₅₀ values. Compound A exhibited induction behavior similar to celastrol at most time points tested. Both compounds activated HSF1 stress granules for up to six hours after heat shock, strongly suggesting that they may maintain or stabilize the active conformation of HSF1 for continuous induction of HSPs. HSP70 signals peaked one hour after the heat shock and maintained a relatively high level (˜25% positively stained cells) even after 6 hours of the heat shock. The long-lasting expression of HSP70 provides an extended protection window for restoring mis-folded proteins.

### Example 2

### Evaluation of Screening Hits from HSF1/HSP70 in OGD Stress

These experiments is to test the hits from the MaCRA HSF1 activator screening in a secondary oxygen glucose deprivation (OGD) assay for cytoprotective effects of test compounds on SHSY5Y cells. SHSY5Y cells were plated at a density of 25,000 cells/well in 96-well plates pre-coated with collagen I (BD Biosciences, San Diego, Calif.) and grown for 16-24 hours in complete medium (Neural Basal Medium, Invitrogen, Carlsbad, Calif.). For the induction of OGD, cells were washed twice in pre-deoxygenated medium with no glucose or serum. Selected compounds at a desired concentration were added to the cells one hour before stress, and the plates were placed in modular incubator chambers (Billups-Rothenberg, Del Mar, Calif.). The chambers were flushed with a gas mixture of 95% N₂/5% CO₂ at a flow rate of 10 L/min for 30 min at room temperature. The residual oxygen (O₂) concentration was monitored using a special O₂ electrode with the final concentration less than 1%. After flushing, the chambers were sealed and maintained in a 37° C. incubator for 28 hours. Following OGD experiments, immuno-staining was performed to confirm the induction of HIF1α, an indicator of insufficient oxygen or hypoxia. All liquid handling procedures were performed using Sciclone ALH3000 (Caliper Life Science, Hopkinton, Mass.) to achieve better reproducibility. Cell viability was measured using an MTS assay (see below). The OGD experiment showed significant cytoprotective effects for cells treated with compound A when compared to the DMSO (control) treated samples as illustrated in FIG. 7.

### Example 3

### Evolution of Screening Hits from HSF1/HSP70 in Rotenone Model

This experiment tests the hits from the MaCRA HSF1 activator screening in a secondary rotenone assay for cytoprotective effects of test compounds on SHSY5Y cells. The rotenone model of Parkinson's disease is an in vitro system for study of protein aggregation induced cytotoxicity. It has been reported that mitochondria inhibitor rotenone can significantly increase α-synuclein expression which eventually becomes cytoplasmic inclusions similar to Lewy bodies. See Greenamyre et al., Parkinsonism Relat. Disord., Suppl 2:S59-S64 (2003). Therefore, this in vitro system may be adopted to evaluate cytoprotective effects of the HSF1/HSP70 MaCRA screening hits as carried out essentially as described in Sherer et al., J. Neuroscience, 23(34):10756-10764, 2003. The data show that more than 42% of SH-SY5Y cells were killed when 100 nM of rotenone was applied for 24 hours. However, SH-SY5Y cells pretreated with 2.5 μM of Compound A resulted in a 29% increase of cell viability compared to that of DMSO control treated cells. See FIG. 8. In summary, the small molecule HSF1/HSP70 amplifier identified from our HSF1/HSP70 screening can rescue cells from two different stress conditions with cytoprotective benefits, possibly through the mechanism of HSF1/HSP70 amplification.

### Example 4

### Development of MaCRA Assay with Submaximal Heat Stress Conditions

Experiment 1: These experiments were performed to optimize assay parameters, such as temperature of heat shock and time of recovery. HeLa cells were treated with 0.33% DMSO in a 96-well plate for assay evaluation (see Example 4, Experiment 3 below). The samples underwent heat shock at 39° C. for 2 hours with no recovery time and a separate group of samples (96-well plate) underwent heat shock at 41° C. for 2 hours with no recovery time. Celastrol served as the positive control. As shown in FIG. 9, a number of positive hits were detected when heat shock was performed at 41° C. for 2 hours with no recovery time compared to heat shock at 39° C.

Experiment 2: HeLa cells were pretreated with 0.33% DMSO and samples were treated with one of four heat shock conditions: 1) 43° C. for 2 hours with a 2 hour recovery time; 2) 43° C. for 2 hours with no recovery time; 3) 43° C. for 1 hour with no recovery time; and 4) 43° C. for 1 hour with 2 hours recovery time. As shown in FIG. 10, the CV values for HSP70 granule count are greater than 25%, which are not well suited for quantification. The data also show that heat shock (43° C. for 1 hour) induced an average of 6.83 HSF1 stress granules per nucleus with no recovery time compared to 6.38 HSF1 stress granules per nucleus with 2 hour recovery time. Both values for HSF1 stress granule count in DMSO treated samples are too close to that of positive control treated sample (6.56) thus suggesting that heat shock conditions of 43° C. at 1 or 2 hours with or without recovery time are not optimal conditions for compound screening.

Experiment 3: HeLa cells were seeded in Costar 96-well assay plates (Costar 3904) at a density of 8,000 cells/well approximately 16 to 24 hours before compound treatment. Subsequently, the cells were treated with DMSO. The overall dilution of compound in DMEM was 200-fold, with a final concentration of 30 μM for screening and a serially diluted concentration ranging between 10 μM and 0.1 μM (10 assayed points) for EC₅₀ determination (final DMSO concentration is 0.3% v/v). Heat shock was carried out at 41° C. for 2 hours with no recovery time. Immediately following heat shock, 50 μL of 16% para-formaldehyde was mixed with the culture medium (total volume at 150 μL) to a final concentration of 4%. The plates were incubated at room temperature for 30 minutes before washing with PBS. Permeablization of cellular membrane was achieved using 0.2% Triton X-100 in PBS for 30 minutes. After washing three times with PBS, 80 μL of 5% FBS/PBS was applied to the plate at room temperature for one hour. For antibody staining, a 1:500 dilution of anti-HSF1 and anti-HSP antibody in 1% FBS/PBS was added to the plates. The plates were incubated at room temperature for two hours or at 4° C. overnight. Finally, a mixture of FITC or rhodamine labeled secondary antibody and DAPI were added into the plates at a final concentration of 1:5000 (for DAPI at 5 mg/mL), 1:500 (for FITC/rhodamine labeled anti-rabbit secondary antibodies). After one hour at room temperature, the plates were washed with PBS and stored at 4° C.

Image acquisition and analysis were performed using an INcell 1000 (GE Healthcare, Piscataway, N.J.) integrated with a Twister II (Caliper Life Science) for automated plate delivery. The setting for image acquisition was three images captured per well at 500 ms for DAPI and 100 ms for FITC or rhodamine as described previously (20). Image analysis was carried out using Multi Target Analysis module from Workstation3.6. Algorithms for HSF1/HSP70 granule count, granule area and nuclear intensity CV were established and optimized according to assay conditions and manufacture instructions. EC₅₀ values and curve fitting were performed using Prism 4.0 (GraphPad Software, San Diego, Calif.) with non-linear regression analysis.

Positively-stained cells were then determined using granule count assays. FIG. 11 shows the HSF1 and HSP70 granule count evaluation this experiment.

Experiment 4: HeLa cells in a 96 well plate format (See Experiment 3 above) were pretreated with 0.33% DMSO and underwent heat shock at 41° C. for 2 hours with no recovery time. Positively stained HSF1 and HSP70 cells were then measured using granule intensity CV and granule area. The results of this experiment are illustrated in FIG. 12. Also, Table 1 summarizes the data in tabular form, showing the CV values for HSF1 granule variable, HSP70 granule variable, HSF1 intensity CV variable and HSP70 granule area when cells are exposed to a 41° C. elevated temperature stress for 2 hours with no recovery period.

### Example 5

### Cytoprotection and Cytotoxicity—Secondary Assays

These experiments were carried out to determine whether the effects seen in HSF1/HSP70 induction can be translated to cytoprotection. WEHI or HEK293 cells at a density of 15,000 cells/well were treated with the screening compounds for 72 hours. Taxol (500 nM) and staurosporine (500 nM) were used as positive controls while DMSO was used as a negative control. After 72 hours, cell viability was measured with MTS/PES (a substrate for mitochondrial dehydrogenase which is active only in viable cells). 1050 values were determined for compounds that induced cytotoxicity. (See FIGS. 17A and 17E.)

The MG-132 assay was used as a secondary assay to determine whether the effects seen in HSF1/HSP70 induction can be translated to cytoprotection. SK-N-SH cells at a density of 12,000 cells/wall were treated with a compound. After 30 minutes, 5 μM of MG-132 was added to the cells and incubated for 24 hours. The positive and negative controls for this experiment were CYT492 and DMSO, respectively. After 24 hours, cell viability was measured with ATPlite according to manufacturer (Perkin-Elmer) specifications. The EC₅₀ values were determined for the compounds that protected cells from MG-132 induced cell death. (See FIGS. 17A, 17D and 17E).

### Example 6

### Tunicamycin ER Stress Model

This experiment is to test whether the screening hits from the HSF1/HSP70 assay can protect or rescue tunicamycin treated cells undergoing ER stress. The procedures used to generate the data shown in FIG. 13 were performed essentially as described in Boyce et al., Science, 307:935-939 (2005) or Yung et al., The FASEB Journal, 21:872-884 (2007). In particular, Compound B at a final concentration of 10 μM was added to the cell culture at various time points. PC12 cells were induced with 750 μg/mL tunicamycin to induce ER stress. The live cells can be measured with ATPlite (Perkin Elmer, Waltham, Mass.). As shown in FIG. 13, Compound B protects PC-12 cells from tunicamycin induced ER stress.

### Example 7

### Chaperone HSF1 Co-Inducer

This test was carried out to compare the increase of HSF1 granule positive cells in non-stressed cells compared to as a function of increasing concentration of celastrol or Compound A. HeLa cells at 8,000 cells/well were treated with increasing concentrations of celastrol or Compound A (0.78 μM to 35 μM) and incubated for 3 hours at 37° C. 50 μL of 16% para-formaldehyde was mixed with the culture medium (total volume at 150 μL) to a final concentration of 4%. The plates were incubated at room temperature for 30 minutes before washing with PBS. Permeablization of cellular membrane was achieved using 0.2% Triton X-100 in PBS for 30 minutes. After washing three times with PBS, 80 μL of 5% FBS/PBS was applied to the plate at room temperature for one hour. For antibody staining, a 1:500 dilution of anti-HSF1 and anti-HSP antibody in 1% FBS/PBS was added to the plates. The plates were incubated at room temperature for two hours or at 4° C. overnight. Finally, a mixture of FITC or rhodamine labeled secondary antibody and DAPI were added into the plates at a final concentration of 1:5000 (for DAPI at 5 mg/mL), 1:500 (for FITC/rhodamine labeled anti-rabbit secondary antibodies). After one hour at room temperature, the plates were washed with PBS and stored at 4° C.

Image acquisition and analysis were performed using an INcell 1000 As shown in FIG. 14 Compound A does not significantly stimulate HSF1 positive granule formation in non-stressed cells, in contrast to celastrol.

### Example 8

### Counter Screen HSP90 ATPase Assay for Monitoring Compound Effects on HSP90 Inhibition

This experiment was carrier out to test whether certain selected compound hits from the MaCRA screens inhibit HSP90 ATPase activity. 2.5 μg of HSP90 (purified from Sf9 cells) were treated with 10 μM of radicicol and 50 μM of compounds A-G respectively for 3 hours at 37° C. ATPase activity was measured using an ADP quest kit from DiscoveRx (Fremont, Calif.). As shown in FIG. 15, various compounds identified as hits from the MaCRA screens described above do not significantly inhibit the ATPase activity of HSP90. Thus, their effects on HSF1 and HSP70 positive granule formation are independent of HSP90 inhibition.

### Example 9

### Screening Strategy for Identifying HSF1+HSP+Co-Inducers

Based on previously described experiments detailed above 4,000 compounds were screened using a primary HSF1/HSP70 granule assay and secondary MG-132 and MTS assays to identify cytoprotection and cytotoxicity, respectively. Multi-dimensional analysis of the data was performed using Spotfire DecisionSite (TIBCO Spotfire, Somerville, Mass.) with cutoff values for HSF1 granule positive cells % above 20% (HSF1+), HSP granule positive cells % above 30% (HSP+), increase of viable cells % in MG132 assay above 30%, and inhibition % in MTS assay below 20% The data for each screen is shown in FIGS. 17B, 17C and 17D. FIGS. 17A and 17E represent multidimensional compilations of data obtained from screening the 4000 compounds.

Tables 2-4 are summary tables with data from select compounds identified in primary and secondary assays according to the methods of the invention separated into compounds which fall in the HSF1+HSP+(A), HSF1−HSP+(B), and HSF1−HSP− (C) categories, as described above.

### Example 10

### Conversion to 384 Well Format with Celastrol Control

HeLa cells were seeded in ViewPlate-384 assay plates (Part No. 6007460, PerkinElmer) at a density of cells/well approximately 16 to 24 hours before compound treatment. Subsequently, the cells were treated with celastrol (control) or screening compounds. The overall dilution of compound in DMEM was 200-fold, with a final concentration of 30 μM for screening and a serially diluted concentration ranging between 10 μM and 0.1 μM (10 assayed points) for EC₅₀ determination (final DMSO concentration is 0.3% v/v). Immediately following heat shock at 43° C. for 2 hours with no recovery time, 25 μL of 16% para-formaldehyde was mixed with the culture medium (total volume at 75 μL) to a final concentration of 4%. The plates were incubated at room temperature for 30 minutes before washing with PBS. Permeablization of cellular membrane was achieved using 0.2% Triton X-100 in PBS for 30 minutes. After washing three times with PBS, 20 μL of 5% FBS/PBS was applied to the plate at room temperature for one hour. For antibody staining, a 1:500 dilution of anti-HSF1 and anti-HSP antibody in 1% FBS/PBS was added to the plates. The plates were incubated at room temperature for two hours or at 4° C. overnight. Finally, a mixture of FITC or rhodamine labeled secondary antibody and DAPI were added into the plates at a final concentration of 1:5000 (for DAPI at 5 mg/mL), 1:500 (for FITC/rhodamine labeled anti-rabbit secondary antibodies). After one hour at room temperature, the plates were washed with PBS and stored at 4° C.

Image acquisition and analysis were performed using an INcell 1000 (GE Healthcare, Piscataway, N.J.) integrated with a Twister II (Caliper Life Science) for automated plate delivery. The setting for image acquisition was three images captured per well at 500 ms for DAPI and 100 ms for FITC or rhodamine. Image analysis was carried out using Multi Target Analysis module from Workstation3.6. Algorithms for HSF1/HSP70 granule count, granule area and nuclear intensity CV were established and optimized according to assay conditions and manufacture instructions. EC₅₀ values and curve fitting were performed using Prism 4.0 (GraphPad Software, San Diego, Calif.) with nonlinear regression analysis. The results of this screen are illustrated in FIG. 18.

### Example 11

### HSF1 Knockdown Examples

Experiment 1: HeLa cells were transfected with 25 nM of HSF1 siRNA, scramble siRNA and transfection control for 48 hours followed by heat shock at 43° C. for 2 hours or non-heat shock treatment. Western blot experiments verified the knockdown of HSF1 and scramble with GAPDH as a loading control. (See FIG. 19). The HSF1 and HSP70 expression is reduced as indicated by the bars in the bar chart.

Experiment 2: HeLa cells were transfected with 25 nM of HSF1, siRNA, scramble siRNA and allowed to incubate for 48 hours. The cells were treated with 25 μM of Compound B or a DMSO control and subjected to heat shock treatment for 41° C. for 2 hours or non-heat shock treatment. Immunocytochemical experiments were performed for staining HSF1 granules (See Zhang et al., J. Biomol. Screen, “High Content Image-Based Screening for Small Molecule Chaperone Amplifiers in Heat Shock”, In Press, (2008)). Image acquisition was done with INcell 1000 with a 10× object and is shown in FIG. 20.

Experiment 3: HeLa cells were transfected with 25 nM of HSF1 or scramble siRNA (non-target). Immunocytochemical experiments were performed for staining HSF1 granules. (supra). Image acquisition was carried out with an INcell 1000 with a 10× object. Cell count was obtained with a Multi-Target Analysis algorithm in the INcell 1000 Workstation software (see FIG. 21). Table 5 shows compiled data from such siRNA knock down experiments using nine independent hits from the HSF1+HSP70+ category that were also identified as co-inducers (amplifiers) of HSF1. As shown in Table 2, each of these compounds is an HSF1-dependent activator.

Experiment 4: SK-N-SH cells were transfected with 10, 25 and 50 nM of HSF1 siRNA, GAPDH siRNA (control) and scramble siRNA (control). The cells were collected 48 hours after siRNA transfection (using the Hiperfect reagent). A Western blot was performed using anti-HSF1 and anti-GAPDH (loading control) (FIGS. 22A and 22B), or anti-HSP70 and anti-GAPDH (FIG. 22C). Image intensity was analyzed using software from Li-Cor with HSF1 intensity from HSF1 siRNA treated samples normalized to scramble siRNA treated samples. FIGS. 22A-B provide an siRNA knockdown of HSF1 in SK-N-SH cells used in the MG-132 assay with 10, 25 and 50 nM of HSF siRNA against GAPDH siRNA (control) and scramble siRNA (control) for 48 h (A) and 72 h (B) and the corresponding Western blot. The Western blot in FIG. 22C illustrates the effects on HSP70 expression after HSF1 knockdown for 48 hours with 10, 25 and 50 nM of HSF1 siRNA compared with GAPDH siRNA and scramble (control) siRNAs.

Experiment 5: SK-N-SH cells were treated with 50 nM HSF1 siRNA and scramble siRNA for 48 hours. CYT2239, CYT2244, CYT2282 or CYT 2532 was added 30 minutes before the treatment of 5 μM of MG-132 for 24 hours. Viable cells were measured with ATPlite kits. HSF1 knockdown was confirmed by immunocytochemistry and high content imaging with the HSF1 nuclear intensity. FIGS. 24A-D show HSF1 dependent cytoprotection of SK-N-SH cells in the MG-132 assay when treated with 50 nM HSF1 siRNA and scramble siRNA for 48 h following pretreatment with one of compounds CYT 2239 (FIG. 23A), CYT 2244 (FIG. 23B), CYT2282 (FIG. 23C) or CYT 2532 (FIG. 23D).

### Example 12

### Use of HSF1 Granule Assays to Identify Inhibitors of Cell Stress Response

Experiment 1: HeLa cells were treated with four different combinations (10 nM, 100 nM, 1 μM and 10 μM) of triptolide and subsequently underwent heat shock at 43° C. for 1 to 4 hours. FIG. 24 illustrates the dose dependent inhibition of HSF1 granule formation treated with increasing the concentrations of triptolide (10 nM, 100 nM, 1 μM and 10 μM). HSF1 granule count was measured with 5 granules/nucleus used as the threshold. Similar assay conditions were used to test the effect of various compounds selected using MaCRA methods as described herein.

Experiment 2: HeLa cells were treated with 1 μM of Triptolide and 10 μM of CYT975 (▪), CYT1563 (▴) and CYT1590 () 30 minutes before heat shock. The cells subsequently underwent heat shock at 43° C. for 1 hour with 0, 5 or 7 hours recovery time with total HSP70 nuclear and cell intensity as the threshold. FIG. 25A shows the reduction of HSP70 expression.

Experiment 3: HeLa cells were treated with 1 μM of Triptolide and 10 μM of CYT975 (▪), CYT1563 (▴) and CYT1590 () 30 minutes before heat shock. The cells subsequently underwent heat shock at 43° C. for 2 hours with 0, 4 or 6 hours recovery time with total HSP70 nuclear and cell intensity as the threshold. FIG. 25B shows the reduction of HSP70 expression.

Experiment 4: HeLa cells were treated with 1 μM of Triptolide and 10 μM of CYT975 (▪), CYT1563 (▴) and CYT1590 () 30 minutes before heat shock. The cells subsequently underwent heat shock at 43° C. for 3 hours with 0, 3 or 5 hours recovery time with total HSP70 nuclear and cell intensity as the threshold. FIG. 25C shows the reduction of HSP70 expression.

Experiment 5: HeLa cells were treated with 1 μM of Triptolide and 10 μM of CYT975 (▪), CYT1563 (▴) and CYT1590 () 30 minutes before heat shock. The cells subsequently underwent heat shock at 43° C. for 4 hours with 0, 2 or 4 hours recovery time with total HSP70 nuclear and cell intensity as the threshold. FIG. 25D shows the reduction of HSP70 expression.

### Example 13

### 96-Well Plate Evaluation of Triptolide and Screening Hit

Experiment 1: HSF1 granule formation induced by Triptolide (▪), CYT1563 (▴) and DMSO (♦, control) was evaluated in a 96-well plate format (See Example 4) following heat shock at 43° C. for 2 hours with no recovery time. FIG. 26A illustrates the HSF1 inhibition.

Experiment 2: HSF1 granule formation induced by Triptolide (▪), CYT1563 (▴) and DMSO (♦, control) was evaluated in a 96-well plate format (See Example 4) following heat shock at 43° C. for 4 hours with 4 hours recovery time. FIG. 26B illustrates the HSF1 inhibition.

Experiment 3: HSP70 expression induced by Triptolide (▪), CYT1563 (▴) and DMSO (0, control) was evaluated in a 96-well plate format (See Example 4) following heat shock at 43° C. for 2 hours with 4 hours recovery time. FIG. 26C illustrates the HSF1 inhibition.

Experiment 4: HSP70 expression induced by Triptolide (▪), CYT1563 (▴) and DMSO (♦, control) was evaluated in a 96-well plate format (See Example 4) following heat shock at 43° C. for 4 hours with 4 hours recovery time. FIG. 26D illustrates the HSF1 inhibition.

### Example 14

### Conversion of 96 Well Format to 384 Well Format

HeLa cells were treated with DMSO (♦) or CYT 1563 (10 μM) (▪) and subsequently heat shocked at 43° C. for 2 h with no recovery time using HSF1 granule count in a 384-well plate format (See Example 10). Results including the Z′ values and Signal/Background (S/B) ratio are illustrated in FIG. 27.

The foregoing examples are presented for illustrative purposes only, and are not intended to be limiting. One of skill in the art will recognize that additional embodiments according to the invention are contemplated as being within the scope of the foregoing generic disclosure, and no disclaimer is in any way intended by the foregoing, non-limiting examples.

### EQUIVALENTS

Those skilled in the art will recognize, or be able to ascertain using no more than routine experimentation, numerous equivalents to the compounds, compositions, and methods of use thereof described herein. Such equivalents are considered to be within the scope of the claimed invention and are covered by the following claims.

The contents of all references, patents and published patent applications cited throughout this Application, as well as their associated figures are hereby incorporated by reference in their entirety.

1. A high-throughput method for quantitative measuring of the modulation of heat shock protein (HSP) expression or of transcriptional activity of heat shock transcription factor (HSF), comprising:
exposing a cell to a stress, and
measuring one or more of the following variables: (i) coefficient of variability (CV) of nuclear intensity; (ii) granule area; (iii) granule intensity; and (iv) ratio of granule intensity to background intensity; or
measuring a combination of two or more of the following variables: (i) coefficient of variability (CV) of nuclear intensity; (ii) granule area; (iii) granule intensity; (iv) ratio of granule intensity to background intensity; and (v) granule count.

2. (canceled)

3. A high-throughput method for identifying modulators of heat shock protein (HSP) expression or modulators of heat shock transcription factor (HSF) expression, comprising:
treating a cell with a candidate compound,
exposing the cell to a stress, and
measuring one or more of the following variables: (i) coefficient of variability (CV) of nuclear intensity; (ii) granule area; (iii) granule intensity; and (iv) ratio of granule intensity to background intensity; or
measuring a combination of two or more of the following variables: (i) coefficient of variability (CV) of nuclear intensity; (ii) granule area; (iii) granule intensity; (iv) ratio of granule intensity to background intensity; and (v) granule count,
to determine whether the candidate compound is a modulator of heat shock protein (HSP) expression or a modulator of heat shock transcription factor (HSF) expression.

4-5. (canceled)

6. The method of claim 1, wherein the stress is selected from elevated temperature, heavy metal stress, oxidative stress, oxygen glucose deprivation (OGD), and oxygen deprivation (OD).

7. The method of claim 6, wherein the stress is oxygen glucose deprivation (OGD).

8. The method of claim 6, wherein the stress is elevated temperature and the elevated temperature is from about 39° C. to less than or about 43° C.

9-12. (canceled)

13. The method of claim 1, wherein the combination includes granule count.

14. (canceled)

15. The method of claim 1, wherein the combination is CV of nuclear intensity and granule area, granule intensity, or ratio of granule intensity to background intensity.

16-17. (canceled)

18. The method of claim 1, wherein the cell exposed to stress is from a cancer cell or immortalized cell.

19-22. (canceled)

23. The method of claim 1, wherein the granules are HSP or HSF1 positive granules.

24. (canceled)

25. The method of claim 23, wherein the granules are HSP and HSF1 positive granules.

26. The method of claim 1, wherein the measuring comprises measuring the level of HSP expression in a cell exposed to stress and comparing it to the baseline level of HSP expression in a cell not exposed to the stress to quantitatively measure the change in HSP expression associated with the stress exposure.

27. The method of claim 26, wherein the HSP expression associated with the stress exposure is an increase in HSP expression over the baseline level of expression.

28. The method of claim 26, wherein the HSP expression associated with the stress exposure is a decrease in HSP expression below the baseline level of expression.

29. The method of claim 26, further comprising externally altering the baseline level of HSP expression.

30. (canceled)

31. The method of claim 3, wherein the candidate compound is a polypeptide.

32. The method of claim 3, wherein the candidate compound is a small molecule.

33. The method of claim 3, wherein the candidate compound is a nucleic acid moiety.

34. (canceled)

35. The method of claim 3, wherein the method is for identifying activators of HSP and/or HSF, the stress is elevated temperature, and the elevated temperature is 41° C.±about 0.5 C.

36. The method of claim 3, wherein the method is for identifying inhibitors of HSP and/or HSF, the stress is elevated temperature, and the elevated temperature is 43° C.±about 0.5 C.

37. A modulator identified by the method of claim 3, wherein the modulator is useful for treating a disease, condition, or indication accompanied by a physiological stress.

38. A device for inducing heat shock stress in a plurality of cellular samples by elevated temperature, the device comprising:
a plate, and
a heating source for heating the plate,
wherein the plate is positioned so as to transfer heat uniformly to the plurality of cellular samples.

39-41. (canceled)
